(19) United States
(12) Patent Application Publication
(10) Pub. No.: US 2024/0400549 A1
(43) Pub. Date: Dec. 5, 2024

(54) COMPOUNDS AND THEIR USES FOR TREATING NEURODEGENERATIVE DISEASE

(71) Applicant: Academia Sinica, Taipei City (TW)

(72) Inventors: Jen-Tse Joseph HUANG, Taipei City (TW); Jim-Min FANG, Taipei City (TW); Yi-Juang CHERN, Taipei City (TW); Yu-Ling TSENG, Taichung City (TW); Yung-An HUANG, New Taipei City (TW)

(21) Appl. No.: 18/689,852

(22) PCT Filed: Sep. 7, 2022
(86) PCT No.: PCT/US2022/042814

§ 371 Date: Mar. 6, 2024

Related U.S. Application Data
(60) Provisional application No. 63/241,543, filed on Sep. 8, 2021.

(51) Int. Cl. C07D 417/14; A61K 31/454; A61K 31/4545

(52) U.S. Cl. CPC C07D 417/14; A61K 31/454; A61K 31/4545

(57) ABSTRACT

Disclosed herein are compounds designed and used in the degradation of pathogenic protein aggregates and oligomers, thus these compounds are suitable for use as medicaments for preventing and/or treating neurodegenerative diseases such as amyotrophic lateral sclerosis (ALS), frontotemporal lobar degeneration (FTLD), Alzheimer's disease (AD), and Huntington's disease (HD).

### CROSS-REFERENCE TO RELATED APPLICATIONS

This application claims priority and the benefit of U.S. Provisional Patent Application No. 63/241,543, filed Sep. 8, 2021, the entirety of which is incorporated herein by reference.

### BACKGROUND OF THE INVENTION

### 1. Field of the Invention

The present disclosure relates to compounds and their uses in the degradation of protein aggregates and oligomers that relate to neurodegenerative diseases.

### 2. Description of Related Art

The misfolding proteins involved in neurodegenerative disease receive increasing attention because of aging population. Clinicopathologically, regional aggregations of cytosolic or nuclear proteins are the hallmark of these diseases, and many scientists are committed to eliminating those aggregates within the cells in the past. To establish a suitable biochemical platform for drug evaluation in neurodegenerative disease, the misfolding transactivation responsive (TAR) DNA-binding protein (TDP-43) is selected as the initial protein target of this invention. TDP-43 is a ubiquitously expressed DNA/RNA binding protein implicated in gene transcription, pre-mRNA splicing, and translational regulation. The N-terminus truncated TDP-43 (C-TDP-43) was identified as the major component in the inclusions of amyotrophic lateral sclerosis (ALS) and frontotemporal lobar degeneration (FTLD) patients. Later studies also taught that C-TDP-43 and some peptide fragments from C-TDP-43 formed fibrillary aggregates and/or inclusions with amyloid properties. Currently, there is no cure or treatment for such disease, and significant efforts have been made to identify molecules that may modulate the formation of pathological aggregates of TDP-43.

In view of the foregoing, there exist in the related art, a need for identifying molecules that may modulate the pathological TDP-43 aggregates, such molecules will be potential drug candidates for the manufacture of a medicament for the prophylaxis or treatment of neurodegenerative diseases resulted from the formation of pathological proteins such as TDP-43 aggregates.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

This invention is based on the finding that some novel compounds help facilitating the degradation of TDP-43 aggregates in vitro and in vivo. Accordingly, these compounds are useful for the manufacture of a medicament suitable for the treatment or prophylaxis of a neurodegenerative disease resulted from accumulation of pathological forms of TDP-43.

In one aspect, the invention relates to a compound having the structure of formula (I),

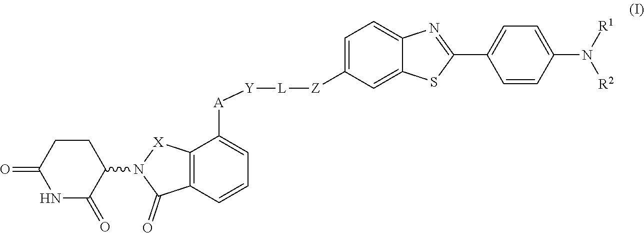

wherein,
X and Y are independently —CH₂—, —CH₂CH₂— or —C═O;
A is —CH₂— or —NH;
Z is —NH, O, or S;
L is —(CH₂CH₂)ₘ—, —(OCH₂CH₂)ₙ—, or

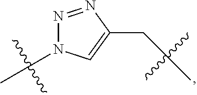

in which m and n are independently an integral between 1 to 8; and
R¹ and R² are independently H or C₁₋₆ alkyl.

In some embodiments, the compound has the structure of formula (I-1)

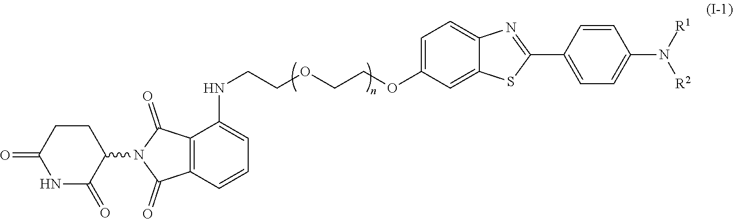

wherein,
n is an integral between 1 to 4; and
R¹ and R² are independently H or C₁₋₆ alkyl.

According to preferred embodiments of the invention, the compound of formula (I-1) is selected from the group consisting of compounds (I-1a) to (I-1d),

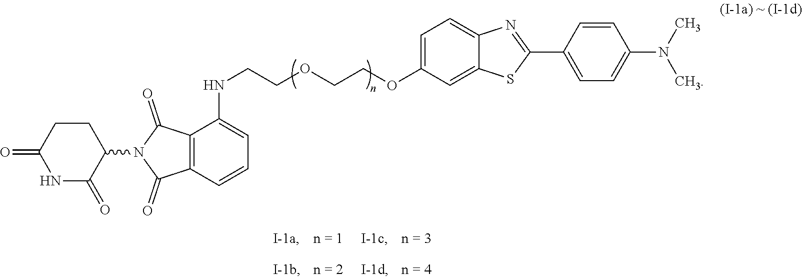

In other embodiments of the invention, the compound has the structure of formula (I-2)

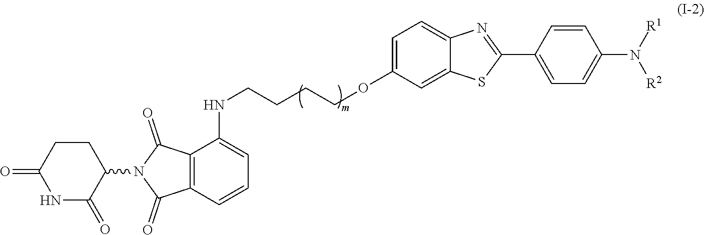

wherein,
m is an integral between 1 to 4; and
R¹ and R² are independently H or C₁₋₆ alkyl.

According to preferred embodiments of the present disclosure, the compound of formula (I-2) is selected from the group consisting of compounds (I-2a) to (I-2d),

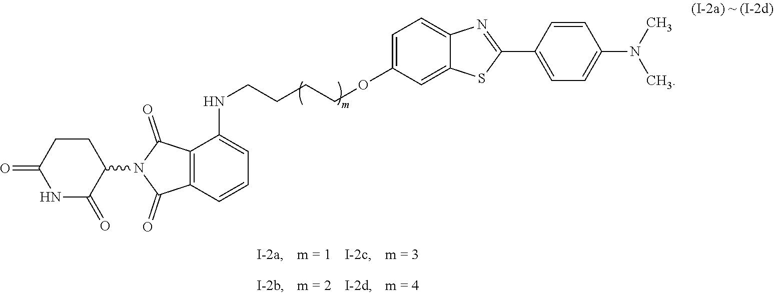

In certain embodiments of the invention, the compound of formula (I) is compound (I-3):

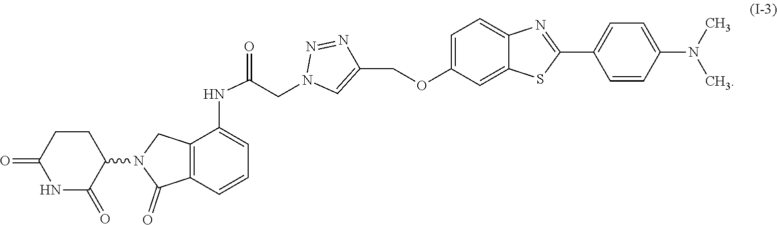 [SMILES: CN(C)c1ccc(-c2nc3ccc(OCc4cn(CC(=O)Nc5cccc6c5CN(C5CCC(=O)NC5=O)C6=O)nn4)cc3s2)cc1]

In another aspect, the invention relates to a compound having the structure of formula (II),

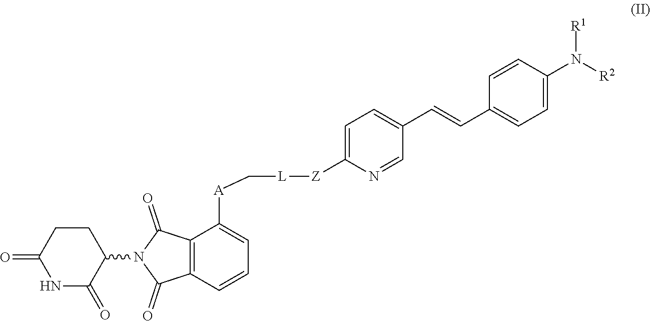

wherein,
A is —CH₂— or —NH;
Z is —NH, O, or S;
L is —(CH₂CH₂)ₘ—, or —(OCH₂CH₂)ₙ—, in which m and n are independently an integral between 1 to 8; and
R¹ and R² are independently H or C₁₋₆ alkyl.

According to some embodiments of the present disclosure, the compound has the structure of formula (II-1),

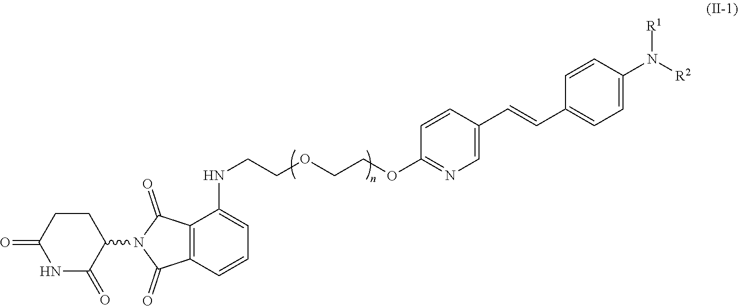

wherein,
n is an integral between 1 to 4; and
R¹ and R² are independently H or C₁₋₆ alkyl.

According to preferred embodiments of the invention, the compound of formula (II-1) is selected from the group consisting of compounds (II-1a) to (II-1d),

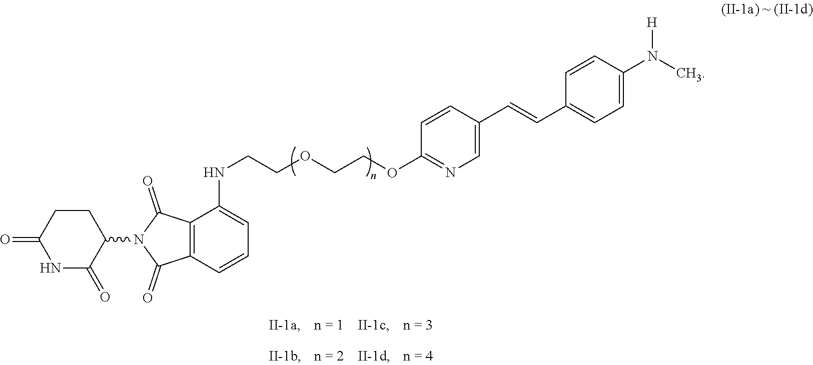

According to further embodiments of the present disclosure, the compound has the structure of formula (II-2),

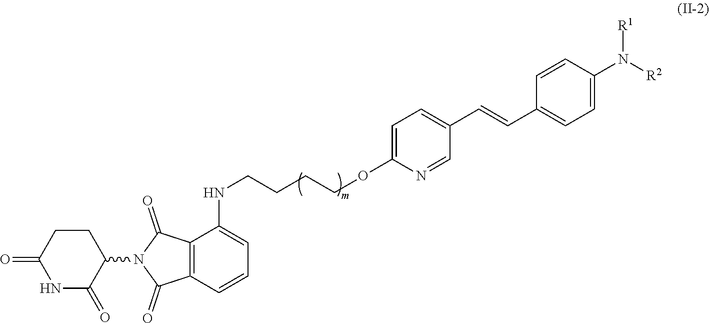

wherein,
m is an integral between 1 to 4; and
R¹ and R² are independently H or C₁₋₆ alkyl.

According to preferred embodiments of the invention, the compound of formula (II-2) is selected from the group consisting of compounds (II-2a) to (II-2d),

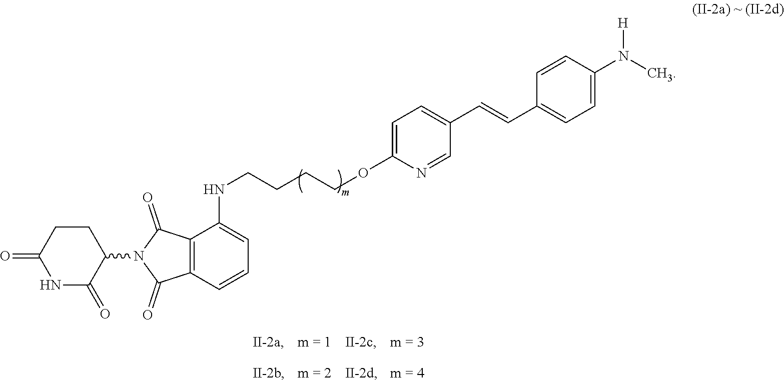

According to embodiments of the present disclosure, the compounds of formula (I) and (II) independently targets an oligomer and an aggregate of a protein that causes a neurodegenerative disease, in which the protein is selected from the group consisting of transactive response (TAR) DNA-binding protein 43 (TDP-43), tau protein, beta-amyloid (A3), and huntingtin.

In a further aspect, the present disclosure provides a pharmaceutical composition suitable for treating a neurodegenerative disease. The pharmaceutical composition comprises the compound of formula (I) or (II); and a pharmaceutically acceptable excipient.

In another aspect, the present disclosure thus pertains to a method for treating a subject having or is at risk of having a neurodegenerative disease. The method comprises administering to the subject an effective amount of the present compound of formula (I) or (II), so as to prevent or ameliorate symptoms associated with the neurodegenerative disease, which is selected from the group consisting of amyotrophic lateral sclerosis (ALS), frontotemporal dementia (FTD), Alzheimer's disease (AD), and Huntington's disease (HD).

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A to 1F illustrates the effect of compounds I-1a to I-1d on C-TDP-43 aggregates or endogenous FL-TDP-43 in Neuro-2a cells in accordance with one embodiments of the present disclosure;

FIGS. 1G to 1J illustrates the effect of compounds II-1b, II-1d, and II-2d on C-TDP-43 aggregates or endogenous FL-TDP-43 in Neuro-2a cells in accordance with one embodiments of the present disclosure;

FIG. 2 illustrates the effect of compounds I-1a to I-1d, II-1b, II-1d, and II-2d on the cell viability of Neuro-2a cells in accordance with one embodiments of the present disclosure;

FIG. 3 illustrates the effect of compounds I-1a to I-1d, II-1b, II-1d, and II-2d on the level of polyQ-expanded HTT_{EX1} protein in accordance with one embodiments of the present disclosure;

FIG. 4 illustrates the effect of compounds I-1a to I-1d, II-1b, II-1d, and II-2d on the level of pathogenic tau protein in accordance with one embodiments of the present disclosure;

FIG. 5 illustrates the effect of compound I-1b in (A) reducing YFP—C-TDP-43 aggregates and (B) improving the motility of the neuronal YFP—C-TDP-43 transgenic C. elegans in accordance with one embodiments of the present disclosure;

FIG. 6 are photos demonstrating similar expressivity of eGFP-C-TDP-43 and mCherry-C-TDP-43 in Neuro-2a cells in accordance with one embodiments of the present disclosure; and

FIG. 7 illustrates the effect of compound I-1b in (A) decreasing the compactness of C-TDP-43 oligomeric intermediates and (B) reducing the high-ordered oligomeric intermediates in Neuro-2a cells in accordance with one embodiments of the present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Unless otherwise indicated, “an effective amount” of a compound is an amount sufficient to provide a therapeutic or prophylactic benefit in the treatment, management or prevention of a disease or condition, or to delay, minimize or prevent one or more symptoms associated with the disease or condition, or its recurrence. An effective amount of a compound is an amount of an agent, alone or in combination with other therapies, which provides a therapeutic or prophylactic benefit in the treatment, management or prevention of the disease or condition. The term “an effective amount” can encompass an amount that improves overall therapy, reduces or avoids symptoms or causes of a disease or condition, or enhances the therapeutic efficacy of another therapeutic agent; or an amount that improves overall prophylaxis or enhances the prophylactic efficacy of another prophylactic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

The term “subject” or “patient” is used interchangeably herein and is intended to mean a mammal including the human species that is susceptible to infection by a virus. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird, and fowl. In a preferred embodiment, the subject is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

### 2. Novel Bifunctional Compounds of the Present Disclosure

The present disclosure is directed to novel compounds targeting misfolded proteins (e.g., TDP-43 aggregates) and facilitating their degradation. Thus, the present compounds may serve as candidate compounds for the development of medicaments for the treatment and/or prophylaxis of diseases or conditions caused by accumulation of misfolded proteins. The novel compounds are described herein.

In one aspect, the present invention relates to a compound of formula (I):

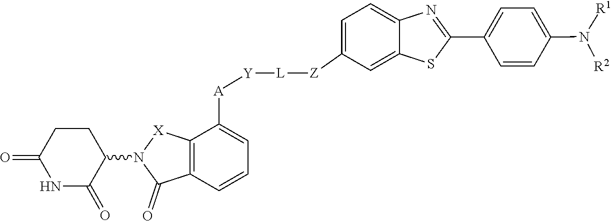

wherein,
X and Y are independently —CH₂—, —CH₂CH₂— or —C═O;
A is —CH₂— or —NH;
Z is —NH, O, or S;
L is —(CH₂CH₂)ₘ—, —(OCH₂CH₂)ₙ—, or

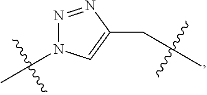

in which m and n are independently an integral between 1 to 8; and
R¹ and R² are independently H or C₁₋₆ alkyl.

According to embodiments of the present disclosure, particular compounds are of formula (I-1),

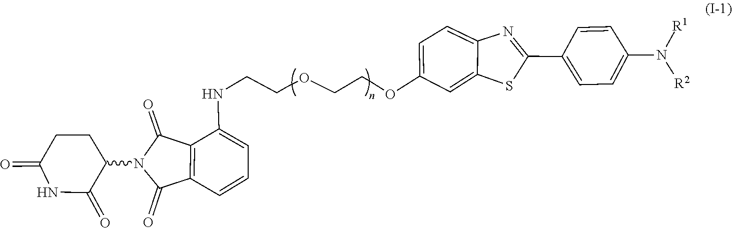

wherein,
n is an integral between 1 to 4; and
R¹ and R² are independently H or C₁₋₆ alkyl.

Exemplary compound of formula (I-1) is selected from the group consisting of compounds (I-1a) to (I-1d),

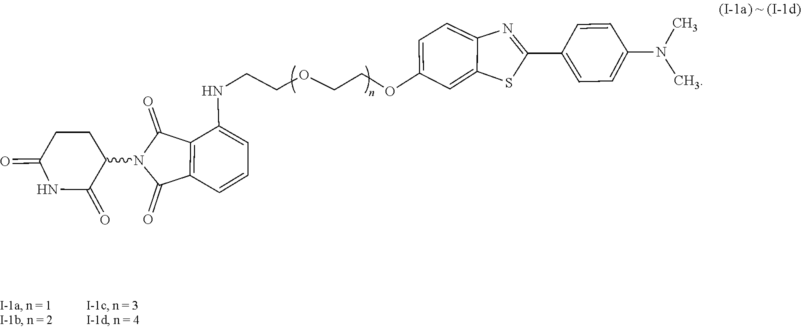

According to other embodiments of the present disclosure, particular compounds are of formula (I-2),

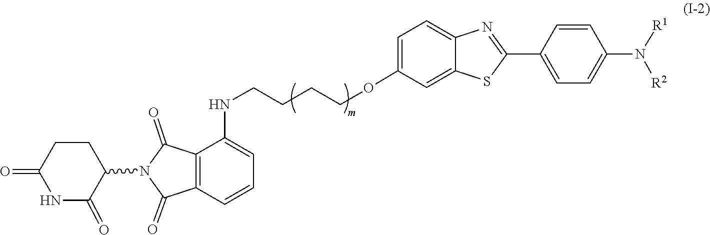

wherein,
m is an integral between 1 to 4; and
R¹ and R² are independently H or C₁₋₆ alkyl.

Exemplary compound of formula (I-2) is selected from the group consisting of compounds (1-2a) to (1-2d),

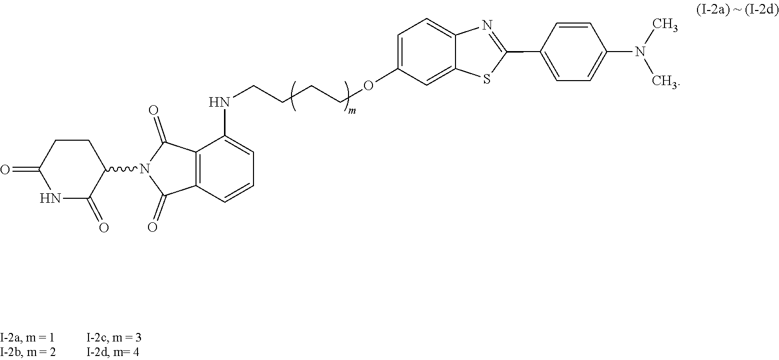

According to certain embodiment of the present disclosure, particular compound is compound (I-3):

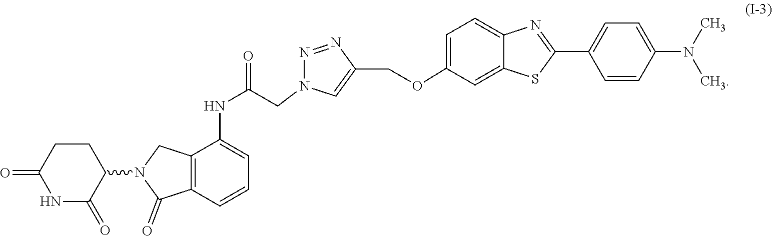 [SMILES: CN(C)c1ccc(-c2nc3ccc(OCc4cn(CC(=O)Nc5cccc6c5CN(C5CCC(=O)NC5=O)C6=O)nn4)cc3s2)cc1]

In another aspect, the present disclosure relates to a compound having the structure of formula (II),

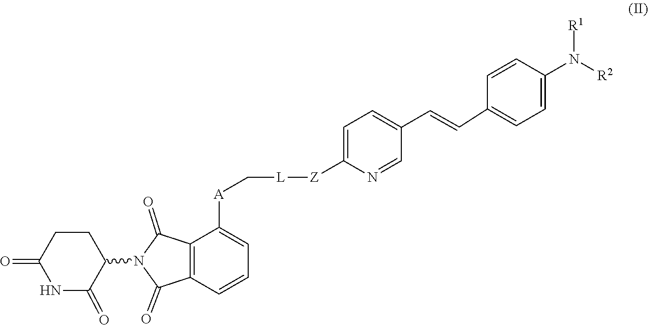

wherein,
A is —CH₂— or —NH;
Z is —NH, O, or S;
L is —(CH₂CH₂)ₘ—, or —(OCH₂CH₂)ₙ—, in which m and n are independently an integral between 1 to 8; and
R¹ and R² are independently H or C₁₋₆ alkyl.

According to embodiments of the present disclosure, particular compounds are of formula (II-1),

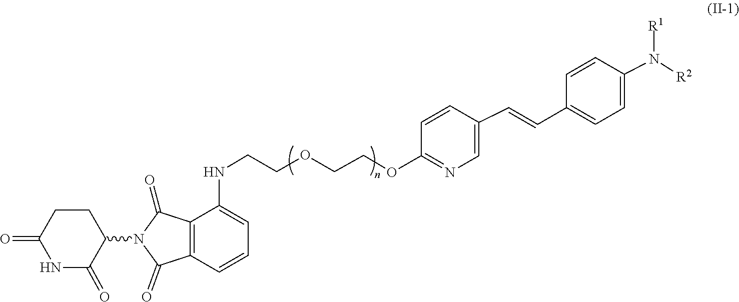

wherein,
n is an integral between 1 to 4; and
R¹ and R² are independently H or C₁₋₆ alkyl.

Exemplary compound of formula (II-1) is selected from the group consisting of compounds (II-1a) to (II-1d),

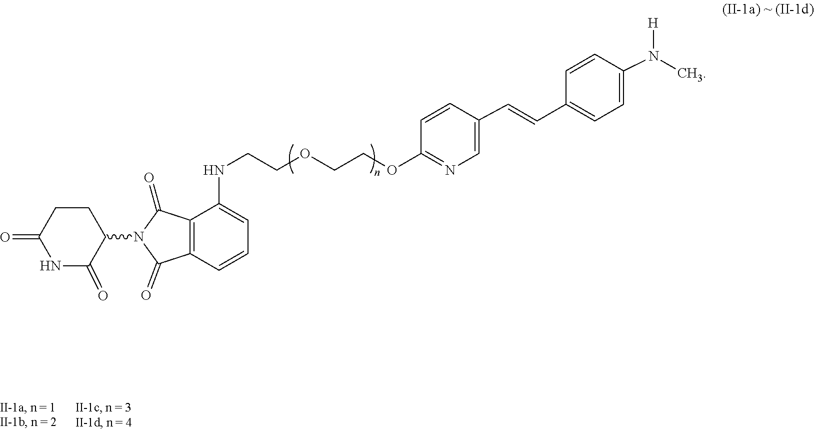

According to further embodiments of the present disclosure, particular compounds are of formula (II-2),

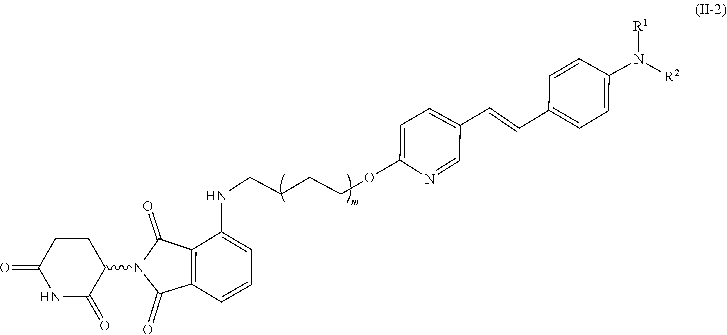

wherein,
m is an integral between 1 to 4; and
R¹ and R² are independently H or C₁₋₆ alkyl.

Exemplary compound of formula (II-2) is selected from the group consisting of compounds II-2a) to (II-2d),

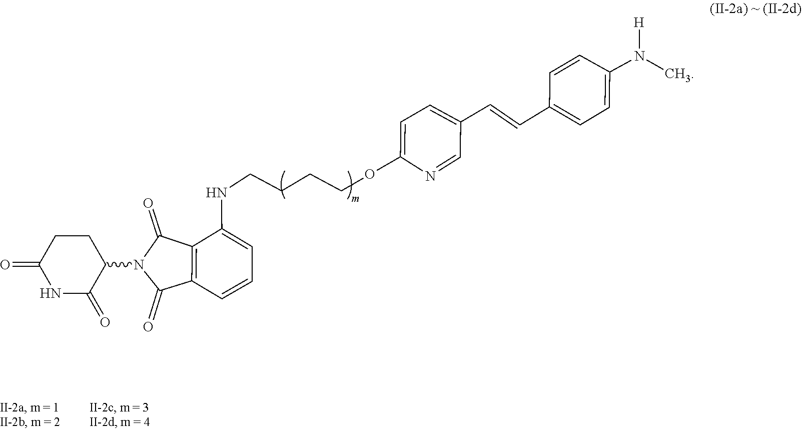

The compound of the present disclosure may be prepared in accordance with procedures described in the working examples. All stereoisomers of the present compounds, such as those which may exist due to asymmetric carbons on the R substituents of the compound of formula (I) or (II) including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

### 3. Method of Use

The present invention encompasses a method for the treatment or prophylaxis of a subject having or is at risk of having a neurodegenerative disease. The method comprises the step of administering an effective amount of the present compound of formula (I) or (II) to the subject, so as to alleviate or ameliorate symptoms associated with the neurodegenerative disease by reducing the accumulated misfolded protein (e.g., TDP-43, huntingtin, tau protein and the like) in the subject.

Advantageously, the compound of formula (I) or (II) is administered in an amount of 0.01 mg to 5,000 mg per day, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460,470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, and 5,000 mg. More preferably, the compound of formula (I) or (II) is administered in an amount of 0.1 mg to 2,500 mg per day, such as 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460,470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, 1,000, 1,100, 1,200, 1,300, 1,400, 1,500, 1,600, 1,700, 1,800, 1,900, 2,000, 2,100, 2,200, 2,300, 2,400, and 2,500 mg. Most preferably, the compound of formula (I) or (II) is administered in an amount of 1 mg to 1000 mg per day, such as 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, and 1,000 mg.

The compound of formula (I) or (II) may be administered one or more times per day, such as twice, or thrice per day, by dividing the daily dose mentioned above for two- or three-times administration. For example, a daily dose of 1,000 mg will be administered in a proportion of two doses of 500 mg each. It is understood that each dose may consist of one or more pharmaceutical forms, for example, a dose of 500 mg may consist of two pharmaceutical forms of 250 mg each.

The amount, route of administration and dosing schedule of the compound of formula (I) or (II) will depend upon factors such as the specific indication to be treated, prevented, or managed, and the age, sex and condition of the patient. The roles played by such factors are well known in the art, and may be accommodated by routine experimentation.

According to one preferred embodiment, in the formula (I), X is —C═O, Y is —CH₂CH₂—, A is —NH, Z is O, L is —(OCH₂CH₂)ₙ—, and n is 2, and administration of the compound results in reduced level of misfolded protein (e.g., TDP-43 aggregates, huntingtin, tau protein and the like) in the subject.

Exemplary diseases treatable by the present method include, but are not limited to, amyotrophic lateral sclerosis (ALS), frontotemporal lobar degeneration (FTLD), Alzheimer's disease (AD), and Huntington's disease (HD).

### 4. Pharmaceutical Formulation

The present disclosure also encompasses pharmaceutical compositions suitable for the treatment and/or prophylaxis of a neurodegenerative disease.

In some embodiments, the present compound of formula (I) or (II) is formulated with one or more pharmaceutically acceptable excipients to form a pharmaceutical composition according to techniques known to those skilled in the art. The compound of formula (I) or (II) is present at a level of about 0.1% to 99% by weight, based on the total weight of the pharmaceutical composition. In some embodiments, the compound of formula (I) or (II) is present at a level of at least 1% by weight, based on the total weight of the pharmaceutical composition. In certain embodiments, the compound of formula (I) or (II) is present at a level of at least 5% by weight, based on the total weight of the pharmaceutical composition. In still other embodiments, the compound of formula (I) or (II) is present at a level of at least 10% by weight, based on the total weight of the pharmaceutical composition. In still yet other embodiments, the compound of formula (I) or (II) is present at a level of at least 25% by weight, based on the total weight of the pharmaceutical composition.

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intra-arterial), or transdermal administration to a patient. Examples of dosage forms include, but are not limited to, tablets; caplets; capsules (e.g., soft elastic gelatin capsules); cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

Similarly, poorly soluble compounds may be incorporated into liquid dosage forms (and dosage forms suitable for reconstitution) with the aid of solubilizing agents, emulsifiers and surfactants such as, but not limited to, cyclodextrins (e.g., α-cyclodextrin or β-cyclodextrin), and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:corn oil), lipids such as egg york phosphatidylcholine (EPC), soybean phosphatidylcholine (SPC), 1,2-dioleoyl-sn-glycero-3-phosphocholine (DOPC), 1,2-distearoyl-sn-glycero-3-phosphocholine (DSPC), cholesterol (CHO), dipalmitoylphosphatidylcholine (DPPC) and PEG-2000. According to one preferred embodiment, the compound of formula (I) (i.e., BO-2590) is incorporated into lipids to form liposomes suitable for oral or parenteral administration.

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art.

### 4.1 Oral Dosage Forms

Pharmaceutical compositions of the present invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or non-aqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Dis-integrants may be incorporated in solid dosage forms to facility rapid dissolution. Lubricants may also be incorporated to facilitate the manufacture of dosage forms (e.g., tablets).

### 4.2 Parenteral Dosage Forms

Parenteral dosage forms can be administered to patients by various routes including, but not limited to, subcutaneous, intravenous (including bolus injection), intramuscular, and intra-arterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: water; aqueous vehicles such as, but not limited to, sodium chloride solution, Ringer's solution, and Dextrose; water-miscible vehicles such as, but not limited to, ethyl alcohol, polyethylene glycol, and polypropylene glycol; and non-aqueous vehicles such as, but not limited to, lipids, corn oil, cottonseed oil, peanut oil, sesame oil, ethyl oleate, isopropyl myristate, and benzyl benzoate.

### 4.3 Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any theory or scheme of action

### Materials and Methods

### Cereblon Binding Assay

A cereblon TR-FRET binding assay was developed by using XL-665-labelled thalidomide (Perkin Elmer) and a specific glutathione S-transferase (GST) antibody labelled with europium cryptate which binds GST-tagged human cereblon/DDB1 protein. This assay could detect competitive ligand that replaces the binding of thalidomide to human cereblon/DDB1 protein. GST-tagged human cereblon and DDB1 protein were co-expressed by using baculovirus expression system. The GST-tagged protein complex was purified by using Glutathione Sepharose (Cytiva LifeSciences) and the purity of the recombinant protein was confirmed by SDS-PAGE. The competitive binding of a given compound was measured by incubating various concentration of compounds with 100 nM cereblon/DDB1 in a buffer containing 50 mM Tris at pH 7.5 and 200 mM NaCl. Final concentration of DMSO was kept at 2.5%. Subsequently, the reaction was added 20 μL of 10 nM XL-665-labelled-thalidomide and 100 nM europium cryptate-labelled GST antibody. All assays were performed in 384-well plates (Geiner Bio-One) and the signals were measured using a Pherastar (BMG) plate reader with excitation at 337 nm and emission at 665 nm/620 nm for detection. The ratio of the acceptor (XL665, 665 nm) and the donor (europium-cryptate, 620 nm) emission signals were used for calculation of EC₅₀ values by using a nonlinear fit model (GraphPad Prism Software). Data are presented as means±standard deviation (n=3).

### Plasmid Construct

The cDNA encoding N-terminal truncated TDP-43 (TDP-43208-414, C-TDP-43) were constructed into pEGFP-C3 vector. To get the mCherry-C-TDP-43 plasmid, C-TDP-43 was subcloned from pEGFP-TDP-43208-414 construct into pcDNA3.1-mCherry vector. Either eGFP or mCherry was fused N-terminally of TDP-43208-414. pEGFP-C3 and pcDNA3.1-mCherry were used as control plasmids for FLIM-FRET experiments.

### Cell Culture and Transfection

Mouse neuroblastoma cell line (Neuro-2a) were cultured in Dulbecco's modified Eagle's medium (DMEM; Gibco) containing 2×10⁻³ M glutamine, 10% heat inactivated fetal bovine serum (FBS), and 100 U mL⁻¹ penicillin-streptomycin (Gibco) at 37° C. in a humidified incubator containing 5% CO₂. Alternatively, Neuro-2a cells were cultured in Dulbecco's modified Eagle's medium (DMEM; Invitrogen, Carlsbad, CA, USA) supplemented with 10% heat-inactivated FBS. The cells were incubated in a humidified incubator at 37° C. with 10% CO₂. Transfection was performed using T-Pro NTR II (Ji-Feng Biotechnology, Taiwan) following the manufacturer's protocol.

The SH-SY5Y-Tau-P301L cell line (SY5Y-Tau) were cultured in DMEM supplemented with 10% FBS and incubated in a humidified incubator at 37° C. with 5% CO₂. To induce the expression of pathogenic Tau (pTau), SY5Y-Tau cells were seeded at 1×10⁵/60-mm plate, cultured for 2-3 days to reach 70% confluence, and treated with doxycycline (1 μg/mL) for 24 hr to induce the expression of pTau.

### Fractionation for Neuro-2a Cells Extract

Extract of C-TDP-43 expressing Neuro-2a cells were centrifuged at 70,000 g at 4° C. for 40 min (Optima™ MAX-XP Ultracentrifuge, Beckman Coulter). The supernatant was carefully transferred (RIPA-soluble) to a new tube and the pellet was washed with RIPA buffer. During the washing step, the pellet tube was centrifuged at 70,000 g at 4° C. for 10 min and the supernatant was carefully removed (more than two times is recommended to remove most of the unwanted RIPA-soluble protein). Lastly, 50 μL 1% sarkosyl buffer (1 g sarkosyl in 50 mL PBS buffer) was added into the pellet tube, followed by intense pipetting to resuspend the pellet (sarkosyl-soluble). The soluble and insoluble samples were further processed for detection by using Western blot.

### AlamarBlue Reduction Assay

Neuro-2a cells were seeded in a 24-well plate at a concentration of 8×10⁴ cells/well and incubated overnight. The attached Neuro-2a cells were then transfected with the eGFP-C-TDP-43 plasmid (1.1 μg) using TurboFect™ transfection reagent (Invitrogen) according to manufacturer's recommendations, and treated with or without 5 μM test compound after 2 h transfection. The cell viability indicator, AlamarBlue (Invitrogen), was added after 42 h of incubation, and the mixture was incubated for another 6 h. Two-hundred L conditional medium was transferred to a 96-well plate, and cell viability was determined by the increased fluorescence intensity (λₑₓ=560 nm, λₑₘ=590 nm).

### Filter Trap Assay and Slot Blot Assay

Neuro-2a cells were seeded in a 6-well plate at a concentration of 2×10⁵ cells/well and incubated overnight. Once the cells were attached, Neuro-2a cells were transfected with the eGFP-C-TDP-43 plasmid (2.2 μg) using Turbofect transfection reagent (Invitrogen) and treated with or without 5 μM test compound after 2 h transfection. After incubation for another 22 h, the transfected cells were harvested by RIPA buffer containing protease inhibitor (Roche) and sonicated on ice for 10 seconds. Extracts were centrifuged at 14000 rpm for 10 min at 4° C., followed by measurement of protein concentration using a bicinchoninic acid (BCA) assay. For filter trap assay, 300 μL sample (100 μg total protein) were passed through 0.2 μm cellulose acetate (CA) membranes (OE66, GE Healthcare) membranes using a 48-well slot-blot apparatus. Aggregated eGFP-C-TDP-43 protein trapped by the CA membranes was determined by immunoblotting. Amersham™ Protein® Nitrocellulose (NC) membranes (pore size 0.1 m, GE healthcare) were applied to slot blot assay for soluble protein lysate analysis.

### In Vitro Protein Binding Assay

Neuro-2a cells were seeded in a 10 cm dish at a concentration of 1×10⁶ (blank control) or 2×10⁶ (mCherry-C-TDP-43 overexpression) cells/mL. After cells were attached, Neuro-2a cells were transfected with the mCherry-C-TDP-43 plasmid (10 μg) using Lipofectamine® 3000 (Invitrogen) and incubated for 24 h. The transfected cells were then harvested with RIPA buffer containing complete protease inhibitor (Roche) and sonicated on ice (10 seconds, two repetitions). Then, different concentration of compound I-1b (0, 5, 10, 20, and 40 μM) was added to each extract (identical protein quantity, 100 μg). The mixtures were gently shaken at 4° C. for 2 h. After that, the mixtures were fractionated and the sarkosyl-soluble part of the mixtures were loaded on NC membrane by applying slot blot assay. The fluorescence of compound I-1b bound to mCherry-C-TDP43 was detected by Typhoon9410 Variable Mode Imager (Amersham BioScience, Piscataway, NJ, USA) (λₑₓ=457 nm, λₑₘ=488 nm).

### Epifluorescence Microscopy

Neuro-2a cells (2×10⁶ cells) were transfected with 1.1 μg of mCherry-C-TDP-43 or eGFP-C-TDP-43 plasmid by TurboFect transfection reagent (Invitrogen). For quantifying the C-TDP-43 aggregates in the presence or absence of compound I-1b or MG132, mCherry-C-TDP-43 transfected cells were treated with 5 μM of compound I-1b after 2 h of transfection. To block proteasome activity, MG132 (2 μM) was added 1 hour before treatment of compound I-1b. After 24 h incubation, the images of fixed cells were taken using microscopy. To monitor the eGFP-C-TDP-43 aggregation in the presence or absence of compound I-1b, eGFP-C-TDP-43 transfected cells were treated with 5 μM of compound I-1b after 2 h of transfection. After another 4 h incubation, time series of images were captured by microscope for another 18 h (a totally 24 h process). Time series of fixed cells epifluorescence images were captured using a NIKON TiE microscope where samples were illuminated with an ultrahigh pressure mercury lamp (130 W) for UV excitation or using a 488 nm laser light source. Filters were used to collect fluorescence emission including excited eGFP (excitation D480/40, dichroic D505LP, emission D535/50) and mCherry (excitation D535/50, dichroic D565LP, emission D590LP) cubes. Cellular images were captured with an Andor iXon3 888 back-illuminated high-sensitivity EMCCD camera. Images were edited and cropped using Nikon NIS element software.

### Frequency-Domain Fluorescence Lifetime Imaging

To study the E_{FRET} of oligomeric intermediates of C-TDP-43, we seeded 2×10⁶ cells/dish of Neuro-2a cells in sterile 35 mm μ-Dish and transfected with either both 0.55 μg eGFP-C-TDP-43 and 0.55 μg mCherry-C-TDP-43 or both 0.55 μg eGFP and 0.55 μg mCherry (negative control) plasmids. After 2 hours, compound I-1b was delivered to the experimental group. After 48 h incubation, the Neuro-2a cells were fixed (4% paraformaldehyde in 15 min and stored in 1×PBS buffer) and further analyzed by Q2 FastFLIM system (ISS Inc.). The Neruo2a cells were monitored and captured under oil-immersion objective observation (Nikon Plan Apo 100×/numerical aperture (NA) 1.4). The eGFP-C-TDP-43 excitation sources came from 488 nm (5 mW) sub-nanosecond modulated pulsed laser at the fundamental frequency of 20 MHz was controlled by ISS VistaVision software. The photon counts of eGFP were collected by GaAs photomultiplier tube (PMT) detector with EM1 filter (530/43 nm bandpass filter). To precisely obtain the lifetime value, the calibration of the system was operated by measuring fluorescein, a fluorophore with a single exponential lifetime around 4 ns in ddH₂O, every time before the measurement.

### FLIM-FRET Data Analysis

The fitting method of the FastFLIM images were described in the “Experimental Section” of a previous publication (He, R. Y et al. Adv. Sci. 2020, 7 (2), 1901165). For the fitting of C-TDP-43 intermediates lifetime, the aggregates and soluble regions were first distinguished by setting the threshold of photon counts of eGFP. Then, the soluble regions were fitted by “2-exponential model” fitting to get the average lifetime of C-TDP-43 oligomeric intermediates. The FRET efficiency of oligomeric intermediates was calculated by the formula:

$${E}_{FRET} = \frac{{τ}_{D} - {τ}_{DA}}{{τ}_{D}} = 1 - \frac{{τ}_{DA}}{{τ}_{D}} ,$$

wherein τ_{D} is donor lifetime of eGFP only, and τ_{DA} denotes lifetime of intermediates. Then, the E_{FRET} of each pixel to form the FRET maps as well as the histogram of E_{FRET} (FIG. 12B) were calculated by ISS Software VistaVision. All of the lifetime values in this study were carefully fitted in a reasonable range with the acceptable qui-square value

### Size Exclusion Chromatography (SEC)

Neuro-2a cells were transfected with eGFP-C-TDP-43 and treated with compound I-1b or both compound I-1b and MG132 after 2 hours. After 48 h incubation, the transfected cells were harvested in 1000 μL of ice-cooled RIPA buffer containing protease inhibitor cocktail (Roche) and sonicated on ice for 1 min. Extracts were centrifuged at 14,000 rpm for 10 min at 4° C. and the protein concentrations were determined using BCA assay. Samples containing 300 μg of total proteins in a volume of 500 μL were filtered with a 0.22 m filter (Millipore) and fractionated on a Superdex 200 10/300 column (GE Healthcare) at a flow rate of 0.3 mL/min. Each fraction (1 mL volume/fraction) was collected and subjected to Western blot and slot blot analysis.

### SDS-PAGE and Western Blot Analysis

Proteins were separated using 12% Tris-glycine SDS-PAGE. Proteins were transferred onto PVDF membrane (Millipore). Blots were blocked with 5% bovine serum albumin (BSA, Sigma) in 0.1% PBST for at least 1 hour. After blocking, blots were subjected to incubation with the primary antibodies TDP-43 (C-terminal) (1:1,000, Proteintech), TDP-43 (1:1,000, Abcam), p-TDP-43 (pS409/410) (1:1,000, Cosmo Bio), GFP (1:1,000, Abcam), All (1:1,000, Invitrogen), GAPDH (1:10,000, GeneTex), or β-actin (1:10,000, GeneTex) in 2% BSA and incubated overnight at 4° C. on a shaker. After washed with PBST, the blots were further incubated with HRP-labelled secondary antibodies (1:3,000, GeneTex) at room temperature for another 1 hour. The blots were washed and developed with electrochemiluminescence (ECL, Millipore). The signals were visualized with luminescence (iBright™ FL1000 instrument, Invitrogen).

Alternatively, cells were lysed with RIPA lysis buffer (150 mM sodium chloride, 1% Nonidet P-40, 0.5% sodium deoxycholate, 0.1% SDS, and 50 mM Tris-HCl, pH 8.0) containing 1× protease inhibitor cocktail and 1× phosphatase inhibitor cocktail to prepare total lysates, which were rotated at 4° C. for 1 h. Protein concentrations were determined with a Bio-Rad protein assay kit (Bio-Rad, Hercules, CA, USA). Protein samples were separated on 8-10% SDS-PAGE gels and electrophoretically transferred onto PVDF membranes (Millipore, Billerica, MA, USA). The membranes were incubated overnight at 4° C. with the following primary antibodies: anti-GAPDH (GeneTex; GTX627408), anti-HTT (Habel; a custom antibody raised against protein translated from the exon 1 of HTT (designated HTT_{EX1}), anti-pTau (MC1, a generous gift from Dr. Peter Davies, the Feinstein Institute for Medical Research, New York, USA), and anti-VCP (BD, East Rutherford, NJ, USA). Immunoreactive bands were detected by enhanced chemiluminescence (ECL; Millipore) and recorded using the UVP ChemiDoc-It Imaging System (Upland, CA, USA).

C. elegans Strains Maintenance and Behavioral Assays

The YFP—C-TDP-43 transgenic strains of C. elegans generated in this study were IW33 [Psnb-l:C-TDP-43₂₁₉₋₄₁₄-YFP (iwIs22)]. The strains of nematodes were maintained with standard procedure and grown at 20° C. as previously described (Zhang, T. et al. Hum. Mol. Genet. 2011, 20 (10), 1952). For all assays, the transgenic young two-day (80 hours) adults were used. For larvae synchronization, the eggs were isolated by lysing gravid adult worms with freshly prepared bleaching solution (0.5 mL 5 M NaOH with 1 mL bleach) and incubated in S buffer (recipe) for overnight. For drug treatments, compound I-1b and/or DMSO were solely or along with MG132 applied to fresh NGM plates prior to installing the synchronized C. elegans. The body bends of the corresponding treatment in a duration of 30 seconds of the various strains were documented through SMZ800N stereomicroscope equipped with a CCD camera (Nikon). A body bend was counted as the head of C. elegans travels across the mid-body in 1×PBS buffer. Then, the bending videos of C. elegans were analyzed by ImageJ with the wrMTrck plugin (Nussbaum-Krammer, C. I. et al. J. Vis. Exp. 2015, (95), 52321). To monitor the effects of compound I-1b on C-TDP-43 accumulation in worms, the confocal images were captured using LSM 780 (Zeiss).

### Statistical Analysis

Statistical analysis was performed using one-way analysis of variance followed by posthoc Tukey's test or two-tailed unpaired t-test. Significance was accepted at p<0.05. All the statistical figures and analysis were done by GraphPad Prism9 software.

### Example 1 Synthesis of the Compounds of Formula (I) or (II)

The compounds of formula (I) or (II) were synthesized in accordance with procedures described in schemes 1 to 5.

### 1.1 Compounds I-1a to I-1d

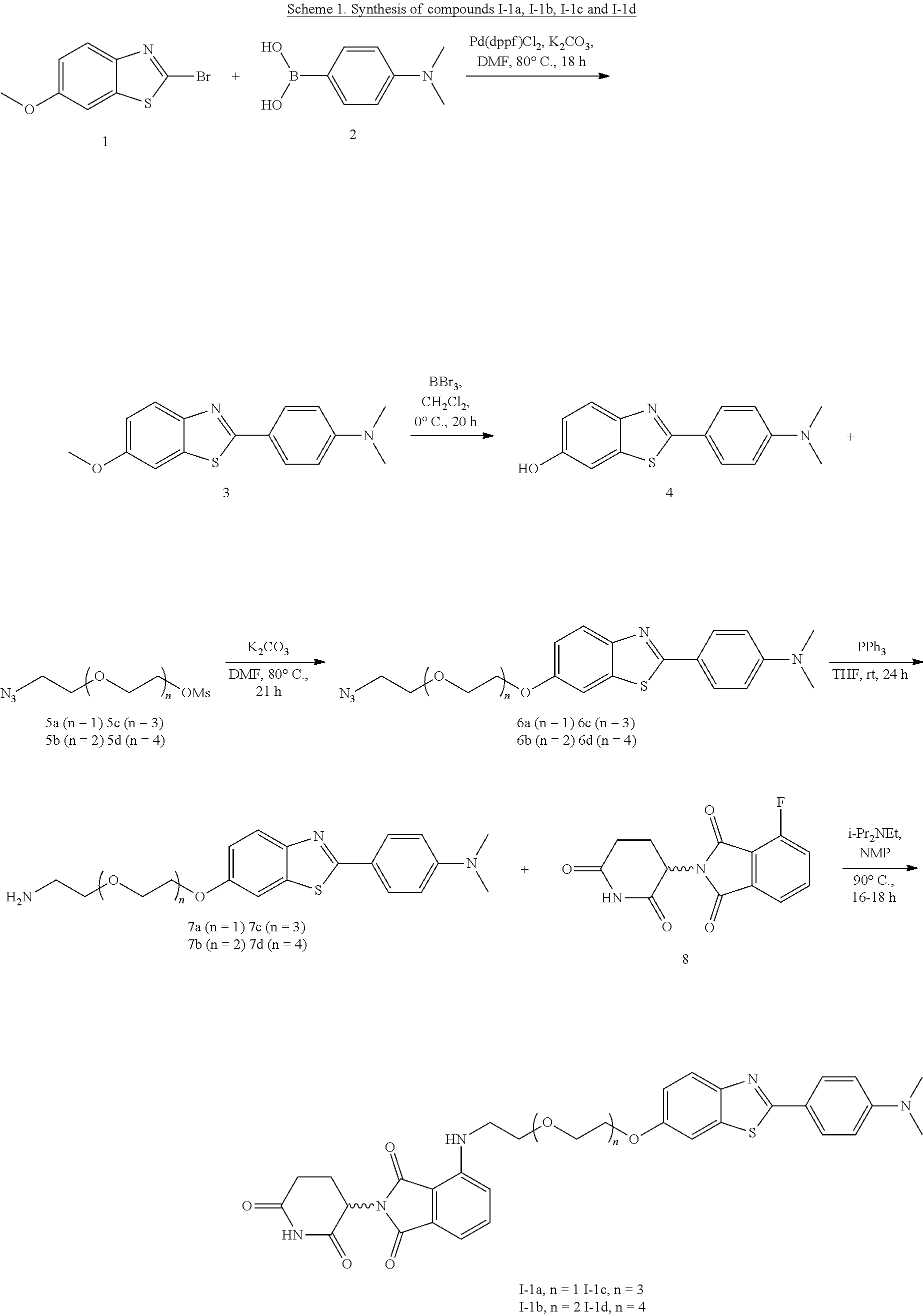

In general, the compound I-1a was synthesized according to Scheme 1. Briefly, the bromo compound 1 and 4-(dimethylamino)phenylboronic acid (2) were subjected to Suzuki coupling reaction in the presence of Pd(dppf)Cl₂ to form anisole 3 as the core structure of TDP43 binder. Anisole 3 reacted with BBr₃ at 0° C. to produce the 6-hydroxy substituted benzothiazole-aniline (BTA) (4). BTA 4 underwent an alkylation reaction with a linker N₃CH₂CH₂(OCH₂CH₂)OMs (5a) in the presence of K₂CO₃ to give compound 6a, followed by reduction of the azido group to give a BTA-linker conjugate 7a bearing a terminal amino group. The reaction of 7a with a pomalidomide analog 8 bearing a fluorine atom at the 4-position of the isoindoline ring was conducted in 1-methyl-2-pyrrolidone (NMP), an aprotic solvent with high polarity, to give the desired product I-1a. Compounds I-1b, I-1c and I-1d having different ethylene glycol linkers were synthesized by the procedures similar to that for I-1a.

### Synthesis of 2-(4-(dimethylamino)phenyl)benzo[d]thiazol-6-ol (Compound 4)

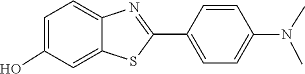 [SMILES: CN(C)c1ccc(-c2nc3ccc(O)cc3s2)cc1]

A solution of 4-(6-methoxybenzo[d]thiazol-2-yl)-N,N-dimethylaniline (compound 3, 1.4 g, 4.9 mmol) in anhydrous CH₂Cl₂ (75 mL) was added BBr₃ (5.61 mL, 60 mmol) dropwisely at 0° C. in an atmosphere of argon. The mixture was stirred at room temperature for 24 h. The reaction was quenched by addition of H₂O, and the solution was adjusted to pH 6-7 by addition of NaOH_{(aq)}. The orange precipitate was collected by vacuum filtration. The crude product was recrystallized from MeOH Et₂O to give pure compound 4 (1.3 g, 98% yield).

C₁₅H₁₄N₂OS; orange solid; mp 227.0-228.0° C.; TLC (EtOAc/hexane=1.2) R_{f}=0.33; IR vₘₐₓ (neat) 3359, 3189, 2920, 2850, 1659, 1634, 1470, 1410, 1133, 734 cm⁻¹; ¹H NMR (400 MHz, DMSO-d₆) δ 9.72 (s, 1H), 7.79 (d, J=8.9 Hz, 2H), 7.72 (d, J=8.8 Hz, 1H), 7.33 (d, J=2.4 Hz, 1H), 6.93 (dd, J=8.8, 2.4 Hz, 1H), 6.79 (d, J=8.9 Hz, 2H), 2.99 (s, 6H). ¹³C NMR (100 MHz, DMSO-d₆) δ 164.2, 155.0, 151.7, 147.4, 135.2 (2×), 127.9, 122.4, 120.6, 115.5, 111.9 (2×), 106.7, 39.5 (2×). ESI-HRMS calcd for C15H15N₂OS: 271.0905, found: m/z 271.0904 [M+H]⁺.

### Synthesis of 2-(2,6-dioxopiperidin-3-yl)-4-fluoroisoindoline-1,3-dione (Compound 8)

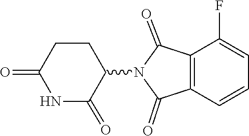 [SMILES: O=C1CCC(N2C(=O)c3cccc(F)c3C2=O)C(=O)N1]

A mixture of 3-fluorophthalic anhydride (100 mg, 0.6 mmol), 2,6-dioxopiperidin-3-amine hydrochloride (99 mg, 0.6 mmol) and NaOAc·3H₂O (98 mg, 0.72 mmol) in AcOH (3 mL) was heated under reflux for 12 h. The mixture was concentrated under reduced pressure, and purified by flash chromatography on a silica gel column with elution of CH₂Cl₂/MeOH (100:1) to give a fluorine-substituted pomalidomide analog 8 (155 mg, 93% yield). The purity of compound 8 was 98.9% as confirmed by HPLC on a silica column (Dikma, 10×250 mm, 10 m particle size), elution: EtOAc/hexane=4:1 at a flow rate of 3.0 mL/min, t_{R}=8.1 min.

C₁₃H₉FN₂O₄; white solid; mp 255.5-257.0° C.; TLC (CH₂Cl₂/MeOH=9:1) R_{f}=0.5; IR vₘₐₓ (neat) 3252, 3107, 2927, 2845, 1717, 1393, 1261, 1199, 746, 597 cm⁻¹; ¹H NMR (400 MHz, DMSO-d₆) δ 11.14 (s, 1H), 7.95 (td, J=7.9, 4.4 Hz, 1H), 7.79 (d, J=7.3 Hz, 1H), 7.73 (t, J=8.9 Hz, 1H), 5.16 (dd, J=12.9, 5.4 Hz, 1H), 2.89 (ddd, J=17.0, 14.0, 5.4 Hz, 1H), 2.66-2.44 (m, 2H), 2.12-2.01 (m, 1H); ¹³C NMR (100 MHz, DMSO-d₆) δ 172.8, 169.8, 166.2, 164.0, 156.9 (d, J_{C-F}=260.9 Hz), 138.1 (d, J_{C-F}=8.1 Hz), 133.5, 123.1 (d, J_{C-F}=19.2 Hz), 120.1, 117.1 (d, J_{C-F}=12.1 Hz), 49.2, 31.0, 21.9; ¹⁹F NMR (376 MHz, DMSO-d₆) δ−115.52 (t, J=4.4 Hz); ESI-HRMS calcd for C₁₃H₉FN₂NaO₄: 299.0439, found: m/z 299.0430 [M+Na]⁺.

### Synthesis of 4-((2-(2-((2-(4-(dimethylamino)phenyl)benzo[d]thiazol-6-yl)oxy)ethoxy)ethyl)amino)-2-(2,6-dioxopiperidin-3-yl)isoindoline-1,3-dione (Compound I-1a)

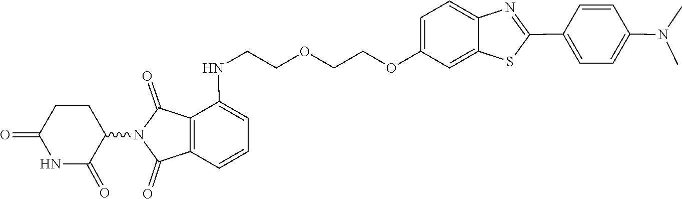 [SMILES: CN(C)c1ccc(-c2nc3ccc(OCCOCCNc4cccc5c4C(=O)N(C4CCC(=O)NC4=O)C5=O)cc3s2)cc1]

To a solution of compound 4 (100 mg, 0.37 mmol) in anhydrous DMF (3 mL) was added K₂CO₃ (102 mg, 0.74 mmol). The mixture was stirred for 30 min at room temperature, and 2-(2-azidoethoxy)ethyl methanesulfonate (compound 5a, 116 mg, 0.56 mmol) was added. The mixture was stirred at 80° C. for 21 h, cooled, and concentrated under reduced pressure. The mixture was extracted with EtOAc and H₂O. The organic phase was dried over MgSO₄, filtered, concentrated under reduced pressure, and purified by flash chromatography on a silica gel column with elution of CH₂Cl₂ to give compound 6a (66 mg, 51% yield).

A THF solution (3 mL) of the above-prepared compound 6a (202 mg, 0.53 mmol) containing a terminal azido group was stirred with PPh₃ (415 mg, 1.6 mmol) and H₂O (30 μL, 1.6 mmol) at room temperature for 24 h. The mixture was concentrated under reduced pressure, and purified by flash chromatography on a silica gel column with elution of CH₂Cl₂/MeOH (10:1) to give the corresponding amino compound 7a (195 mg, 99% yield).

A mixture of compound 7a (181 mg, 0.66 mmol), compound 8 (180 mg, 0.50 mmol) and diisopropylethylamine (DIPEA, 180 μL, 1.01 mmol) in 1-methyl-2-pyrrolidone (NMP) (2.5 mL) was stirred at 90° C. for 18 h. The mixture was extracted with EtOAc and H₂O. The combined organic phase was dried over MgSO₄, filtered, concentrated under reduced pressure, and purified by flash chromatography on a silica gel column with elution of EtOAc/CH₂Cl₂ (1:2) to give the desired compound I-1a (100 mg, 32% yield). The purity of compound I-1a was 96.4% as shown by HPLC on a silica column (Dikma, 10×250 mm, 10 m particle size), elution: EtOAc/hexane=3:1 at a flow rate of 3.0 mL/min, t_{R}=12.2 min. C32H31N₅₀₆S; yellow solid; mp 157.0-158.0° C.; TLC (EtOAc/CH₂Cl₂=1:2) R_{f}=0.63; IR vₘₐₓ (neat) 3359, 3182, 2919, 2849, 1699, 1695, 1657, 1557, 1538, 1471 cm⁻¹; ¹H NMR (400 MHz, CDCl₃) δ 8.15 (s, 1H), 7.97-7.80 (m, 3H), 7.44 (t, J=7.8 Hz, 1H), 7.30 (s, 1H), 7.10-7.00 (m, 2H), 6.90 (d, J=8.5 Hz, 1H), 6.74 (d, J=8.5 Hz, 2H), 6.50 (s, 1H), 4.86 (q, J=5.4 Hz, 1H), 4.19 (t, J=4.3 Hz, 2H), 3.87 (t, J=4.3, 2H), 3.79 (t, J=5.1 Hz, 2H), 3.48 (q, J=5.1 Hz, 2H), 3.03 (s, 6H), 2.86-2.67 (m, 3H), 2.08-2.00 (m, 1H); ¹³C NMR (125 MHz, DMSO-d₆) δ 172.9, 170.2, 169.0, 167.4, 165.5, 156.0, 151.9, 148.3, 146.5, 136.3, 135.2, 132.1, 128.2 (2×), 122.4, 120.5, 117.5, 115.7, 112.0 (2×), 110.8, 109.3, 105.8, 69.0, 68.8, 67.8, 48.6, 41.7 (2×), 31.1, 22.2, 18.6. ESI-HRMS calcd for C32H32N₅₀₆S: 614.2068, found: m/z 614.2031 [M+H]⁺.

### Synthesis of 4-((2-(2-(2-((2-(4-(dimethylamino)phenyl)benzo[d]thiazol-6-yl)oxy)ethoxy) ethoxy)ethyl)amino)-2-(2,6-dioxopiperidin-3-yl)isoindoline-1,3-dione (Compound I-1b)

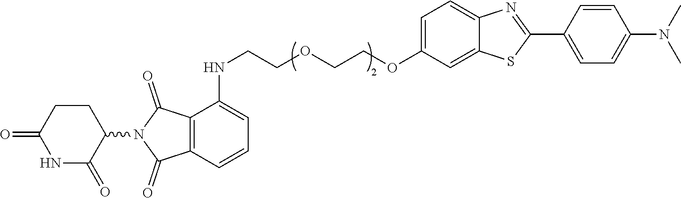

By a procedure similar to that for compound I-1a, the substitution reaction of 8 (74 mg, 0.26 mmol) with 4-(6-(2-(2-(2-aminoethoxy)ethoxy)ethoxy)benzo[d]thiazol-2-yl)-N,N-dimethyl aniline (compound 7b, 54 mg, 0.13 mmol) gave a crude product, which was purified by flash chromatography on a silica gel column with elution of EtOAc/CH₂Cl₂ (1:1) to give the desired compound I-1b (46 mg, 52% yield). The purity of compound I-1b was 97.0% as shown by HPLC on a silica column (Dikma, 10×250 mm, 10 m particle size), elution: EtOAc/hexane=3:1 at a flow rate of 3.0 mL/min, t_{R}=17.9 min. C₃₄H₃₅N₅O₇S; yellow solid; mp 128.0-129.0° C.; TLC (EtOAc/CH₂Cl₂=1:1) R_{f}=0.5; IR vₘₐₓ (neat) 3357, 3197, 2920, 2849, 1653, 1632, 1471 cm⁻¹; ¹H NMR (400 MHz, CDCl₃) δ 8.35 (s, 1H), 7.88 (d, J=8.8 Hz, 2H), 7.82 (d, J=8.9 Hz, 1H), 7.41 (t, J=7.8 Hz, 1H), 7.29 (d, J=2.4 Hz, 1H), 7.01-6.99 (m, 2H), 6.84 (d, J=8.5 Hz, 1H), 6.71 (d, J=8.8 Hz, 2H), 6.44 (t, J=5.4 Hz, 1H), 4.85 (dd, J=12.1, 5.3 Hz, 1H), 4.15 (t, J=4.7 Hz, 2H), 3.86 (t, J=4.7 Hz, 2H), 3.78-3.64 (m, 6H), 3.40 (q, J=5.4 Hz, 2H), 3.01 (s, 6H), 2.87-2.59 (m, 3H), 2.09-2.00 (m, 1H); ¹³C NMR (100 MHz, CHCl₃) δ 171.1, 169.2, 168.4, 167.6, 166.6, 156.2, 151.9, 149.0, 146.7, 136.0, 135.6, 132.4, 128.5 (2×), 122.6, 121.5, 116.7, 115.4, 111.7 (2×), 111.6, 110.2, 105.4, 70.9, 70.7, 69.8, 69.5, 68.1, 48.8, 42.3, 40.14 (2×), 31.3, 22.7. ESI-HRMS calcd for C₃₄H₃₆N₅O₇S: 658.2330, found: m/z 658.2307 [M+H]⁺.

### Synthesis of 4-((2-(2-(2-(2-((2-(4-(dimethylamino)phenyl)benzo[d]thiazol-6yl)oxy)ethoxy) ethoxy)ethoxy)ethyl)amino)-2-(2,6-dioxopiperidin-3-yl)isoindoline-1,3-dione (Compound I-1c)

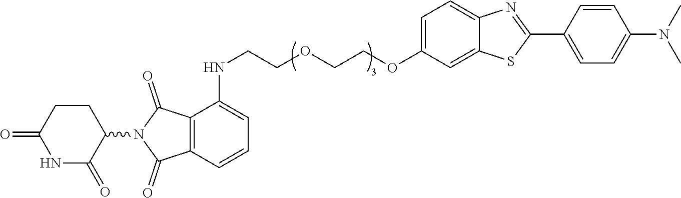

By a procedure similar to that for compound I-1a, the substitution reaction of 8 (88 mg, 0.32 mmol) with 4-(6-(2-(2-(2-(2-aminoethoxy)ethoxy)ethoxy)ethoxy)benzo[d]thiazol-2-yl)-N,N-dimethylaniline (compound 7c, 110 mg, 0.25 mmol) gave a crude product, which was purified by flash chromatography on a silica gel column with elution of EtOAc/CH₂Cl₂ (1:1) to give the desired compound I-1c (70 mg, 40% yield). The purity of compound I-1c was 95.1% as shown by HPLC on a silica column (Dikma, 10×250 mm, 10 m particle size), elution: EtOAc/hexane=9:1 at a flow rate of 3.0 mL/min, t_{R}=15.9 min. C₃₆H₃₉N₅O₈S; yellow solid; mp 87.5-89.0° C.; TLC (EtOAc/CH₂Cl₂=1:1) R_{f}=0.38; IR vₘₐₓ (neat) 3358, 3197, 2919, 2849, 1661, 1645, 1622, 1471, 1407 cm⁻¹; ¹H NMR (400 MHz, CDCl₃) δ 8.55 (t, J=13.5 Hz, 1H), 7.86 (d, J=8.6 Hz, 2H), 7.81 (d, J=8.9 Hz, 1H), 7.39 (t, J=7.8 Hz, 1H), 7.27 (s, 1H), 7.05-6.96 (m, 2H), 6.82 (d, J=8.5 Hz, 1H), 6.68 (d, J=8.6 Hz, 2H), 6.42 (t, J=5.1 Hz, 1H), 4.86 (q, J=4.3 Hz, 1H), 4.13 (t, J=3.9 Hz, 2H), 3.84 (t, J=4.6, 2H), 3.72-3.67 (m, 2H), 3.67-3.59 (m, 8H), 3.38 (q, J=5.2 Hz, 2H), 2.99 (s, 6H), 2.80-2.60 (m, 3H), 2.04 (t, J=6.3 Hz, 1H); ¹³C NMR (100 MHz, CDCl₃) δ 171.2, 169.2, 168.5, 167.5, 166.5, 156.2, 151.9, 148.6, 146.7, 135.9, 135.4, 132.4, 128.5 (2×), 122.5, 121.2, 116.7, 115.4, 111.7 (2×), 111.5, 110.1, 105.4, 70.7, 70.6 (3×), 69.6, 69.4, 68.1, 48.8, 42.3, 40.1 (2×), 31.3, 22.6. ESI-HRMS calcd for C₃₆H₄₀N₅O₈S: 702.2592, found: m/z 702.2589 [M+H]⁺.

### Synthesis of 4-((14-((2-(4-(dimethylamino)phenyl)benzo[d]thiazol-6-yl)oxy)-3,6,9,12-tetraoxatetradecyl)amino)-2-(2,6-dioxopiperidin-3-yl)isoindoline-1,3-dione (Compound I-1d)

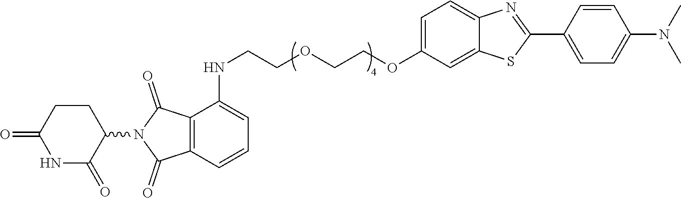

By a procedure similar to that for compound I-1a, the substitution reaction of 8 (102 mg, 0.37 mmol) with 14-((2-(4-(dimethylamino)phenyl)benzo[d]thiazol-6-yl)oxy)-3,6,9,12-tetraoxa tetradecan-1-amine (compound 7d, 90 mg, 0.18 mmol) gave a crude product, which was purified by flash chromatography on a silica gel column with elution of EtOAc/CH₂Cl₂(2:1) to give the desired compound I-1d (89 mg, 65% yield). The purity of compound I-1d was 99.3% as shown by HPLC on a silica column (Dikma, 10×250 mm, 10 m particle size), elution: EtOAc/MeOH=99:1 at a flow rate of 3.0 mL/min, t_{R}=11.3 min. C₃₈H₄₃N₅O₉S; yellow solid; mp 77.5-79.0° C.; TLC (EtOAc/CH₂Cl₂=2:1) R_{f}=0.38; IR vₘₐₓ (neat) 3356, 3197, 2921, 2851, 1653, 1634, 1470, 1456, 1368, 742, 701 cm⁻¹; ¹H NMR (400 MHz, CDCl₃) δ 8.66 (s, 1H), 7.85 (d, J=8.8 Hz, 2H), 7.80 (d, J=8.9 Hz, 1H), 7.40 (t, J=7.8 Hz, 1H), 7.27 (d, J=2.4 Hz, 1H), 7.05-6.96 (m, 2H), 6.83 (d, J=8.6 Hz, 1H), 6.68 (d, J=8.8 Hz, 2H), 6.42 (t, J=5.4 Hz, 1H), 4.85 (q, J=5.2 Hz, 1H), 4.14 (t, J=4.7 Hz, 2H), 3.84 (t, J=4.7, 2H), 3.73-3.67 (m, 2H), 3.67-3.57 (m, 12H), 3.38 (q, J=5.4 Hz, 2H), 2.99 (s, 6H), 2.85-2.60 (m, 3H), 2.07-1.97 (m, 1H); ¹³C NMR (100 MHz, CDCl₃) δ 171.3, 169.1, 168.5, 167.5, 166.5, 156.1, 151.8, 148.7, 146.7, 135.9, 135.5, 132.4, 128.4 (2×), 122.5, 121.3, 116.7, 115.4, 111.7 (2×), 111.5, 110.1, 105.3, 70.8, 70.6, 70.5 (2×), 70.4 (2×), 69.6, 69.3, 68.0, 48.7, 42.2, 40.1 (2×), 31.3, 22.6. ESI-HRMS calcd for C₃₈H₄₄N₅O₉S: 746.2854, found: m/z 746.2888 [M+H]⁺.

### 1.2 Compounds I-2a to I-2d

Compounds I-2a, I-2b, I-2c and I-2d having aliphatic chains of different lengths were synthesized accordance with procedures depicted in Scheme 2.

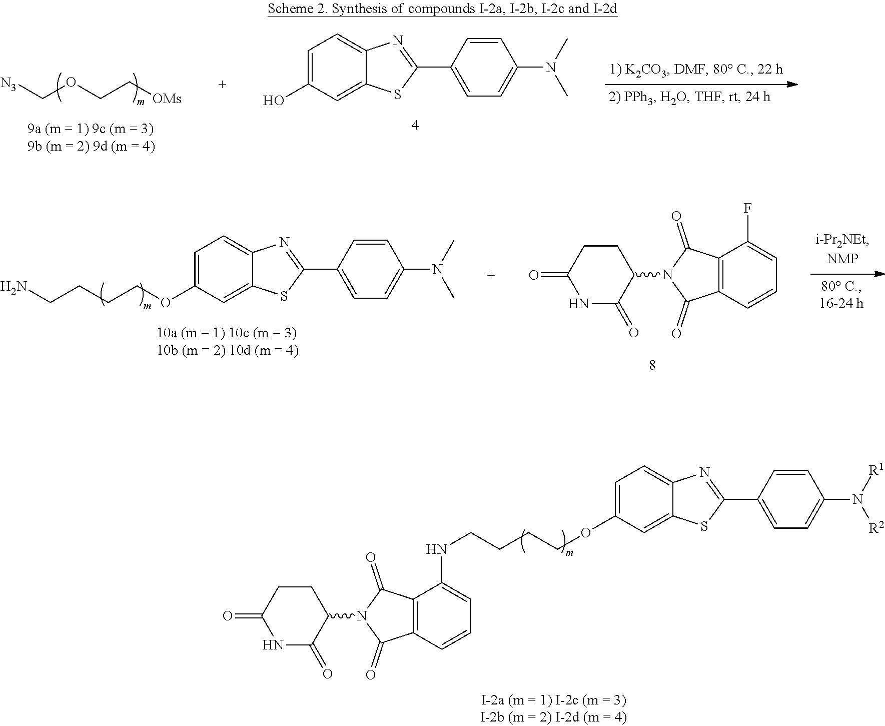

### Synthesis of 6-azidohexyl Methanesulfonate (9b)

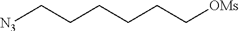 [SMILES: CS(=O)(=O)OCCCCCCN=[N+]=[N-]]

A mixture of 6-bromohexanol (827 mg, 4.5 mmol) and NaN₃ (585 mg, 9 mmol) in DMF (23 mL) was stirred at 80° C. for 16 h. The mixture was concentrated under reduced pressure. The mixture was extracted with EtOAc and H₂O. The organic phase was dried over MgSO₄, filtered, concentrated under reduced pressure to give 6-azidohexanol. A solution of 6-azidohexanol in anhydrous CH₂Cl₂ (9 mL) at 0° C. was added Et₃N (1.9 mL, 14 mmol) and methanesulfonyl chloride (0.7 mL, 9 mmol). The mixture was stirred at room temperature for 2 h. The mixture was extracted with EtOAc and H₂O. The combined organic phase was dried over MgSO₄, filtered, concentrated under reduced pressure and purified by flash chromatography on a silica gel column with elution of EtOAc/hexane (2:3) to give compound 9b (626 mg, 63% yield for two steps). C₇H₁₅N₃₀₃S; colorless liquid; TLC (EtOAc/hexane=2:3) R_{f}=0.53; ¹H NMR (400 MHz, CDCl₃) δ 4.15 (t, J=6.3 Hz, 2H), 3.21 (t, J=6.8 Hz, 2H), 2.93 (s, 3H), 1.69 (t, J=6.7 Hz, 2H), 1.54 (t, J=6.7 Hz, 2H), 1.41-1.32 (m, 4H); ¹³C NMR (100 MHz, CDCl₃) δ 69.7, 51.0, 37.1, 28.8, 28.4, 25.9, 24.8. ESI-HRMS calcd for C₇H₁₅N₃NaO₃S: 244.0726, found m/z 244.0726 [M+Na]⁺.

### Synthesis of 4-(6-((6-aminohexyl)oxy)benzo[d]thiazol-2-yl)-N,N-dimethylaniline (10b)

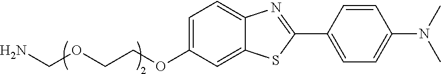

A mixture of compound 4 (200 mg, 0.74 mmol) and K₂CO₃ (204 mg, 1.48 mmol) in anhydrous DMF (7 mL) was stirred for 30 min at room temperature, and mesylated compound 9b (269 mg, 1.11 mmol) was added to the solution. The mixture was stirred at 80° C. for 20 h, cooled, and extracted with EtOAc and H₂O. The combined organic phase was dried over MgSO₄, filtered, concentrated under reduced pressure, and purified by flash chromatography on a silica gel column with elution of CH₂Cl₂/hexane (2:1) to give the alkylation compound (182 mg).

The above-prepared compound (160 mg, 0.4 mmol) in THE (5 mL) was stirred with PPh₃ (213 mg, 2 mmol) and H₂O (0.02 mL, 1.2 mmol) at room temperature for 24 h. The mixture was concentrated under reduced pressure, and purified by flash chromatography on a silica gel column with elution of CH₂Cl₂/MeOH (1:9) to give compound 10b (125 mg, 53% yield for two steps). C₂₁H₂₇N₃₀S; white solid; TLC (CH₂Cl₂/MeOH=9:1) R_{f}=0.05; ¹H NMR (400 MHz, CDCl₃) δ 7.86 (d, J=8.8 Hz, 2H), 7.82 (d, J=8.9 Hz, 1H), 7.26 (d, J=2.3 Hz, 1H), 6.99 (dd, J=8.9, 2.3 Hz, 1H), 6.69 (d, J=8.8 Hz, 2H), 3.96 (t, J=6.5 Hz, 2H), 2.98 (s, 6H), 2.67 (t, J=6.1 Hz, 2H), 1.78 (quin, J=6.9 Hz, 2H), 1.54 (s, 2H), 1.49-1.35 (m, 6H); ¹³C NMR (100 MHz, CDCl₃) δ 166.2, 156.5, 151.8, 148.8, 135.7, 128.4 (2×), 122.6, 121.6, 115.3, 111.7 (2×), 105.1, 68.5, 42.0, 40.1 (2×), 33.6, 29.2, 26.6, 25.8. ESI-HRMS calcd for C₂₁H₂₈N₃OS: 370.1948, found m/z 370.1942 [M+H]⁺.

### Synthesis of 4-((6-((2-(4-(dimethylamino)phenyl)benzo[d]thiazol-6-yl)oxy)hexyl)amino)-2-(2,6-dioxopiperidin-3-yl)isoindoline-1,3-dione (Compound I-2b)

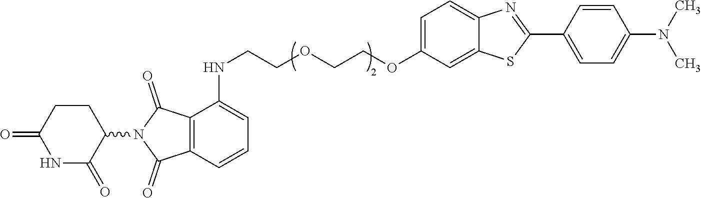

A mixture of compound 10b (50 mg, 0.14 mmol), compound 8 (41 mg, 0.15 mmol) and DIPEA (0.05 mL, 0.27 mmol) in NMP (1 mL) was stirred at 90° C. for 24 h. The mixture was extracted with EtOAc and H₂O. The combined organic phase was dried over MgSO₄, filtered, concentrated under reduced pressure, and purified by flash chromatography on a silica gel column with elution of EtOAc/CH₂Cl₂ (1:9) to give compound I-2b (33 mg, 39%). C₃₄H₃₅N₅O₅S; yellow solid; TLC (EtOAc/CH₂Cl₂=1:9) R_{f}=0.5; ¹H NMR (400 MHz, CDCl₃) δ 8.04 (s, 1H), 7.89 (d, J=9.0 Hz, 2H), 7.84 (d, J=9.2 Hz, 1H), 7.46 (t, J=7.6 Hz, 1H), 7.28 (s, 1H), 7.06 (d, J=7.8 Hz, 1H), 7.01 (dd, J=9.2, 1.6 Hz, 1H), 6.86 (d, J=7.8 Hz, 2H), 6.22 (s, 1H), 4.94-4.78 (m, 1H), 4.00 (t, J=2.2 Hz, 2H), 3.27 (q, J=5.6 Hz, 2H), 3.03 (s, 6H), 2.91-2.67 (m, 3H), 2.09 (t, J=6.0 Hz, 2H), 1.82 (quin, J=6.0 Hz, 2H), 1.70 (quin, J=6.5 Hz, 2H), 1.56-1.45 (m, 4H); ¹³C NMR (100 MHz, DMSO-d₆) δ 172.9, 170.2, 169.0, 167.4, 165.4, 156.2, 152.0, 148.3, 146.5, 136.4, 135.3, 132.3, 128.1 (2×), 122.4, 120.5, 117.3, 115.6, 111.9 (2×), 110.5, 109.1, 105.6, 68.1 (2×), 48.6, 41.8 (2×), 31.0, 28.7, 28.6, 26.1, 25.4, 22.2. ESI-HRMS calcd for C₃₄H₃₆N₅O₅S: 626.2432, found m/z 626.2424 [M+H]⁺.

### 1.3 Compound I-3

Compound I-3 was synthesized using a triazole-containing linker to connect a lenalidomide and a BTA (Scheme 3). The alkylation reaction of the 6-hydroxy substituted BTA (4) with propargyl bromide is carried out to obtain compound 12. The acylation reaction of lenalidomide (13) with 2-iodoacetyl chloride gives compound 15, and the iodine atom is subsequently replaced with azide to give compound 16. Finally, the Cu(I)-catalyzed cycloaddition reaction between alkyne 12 and azide 16 is carried out to yield the compound I-3.

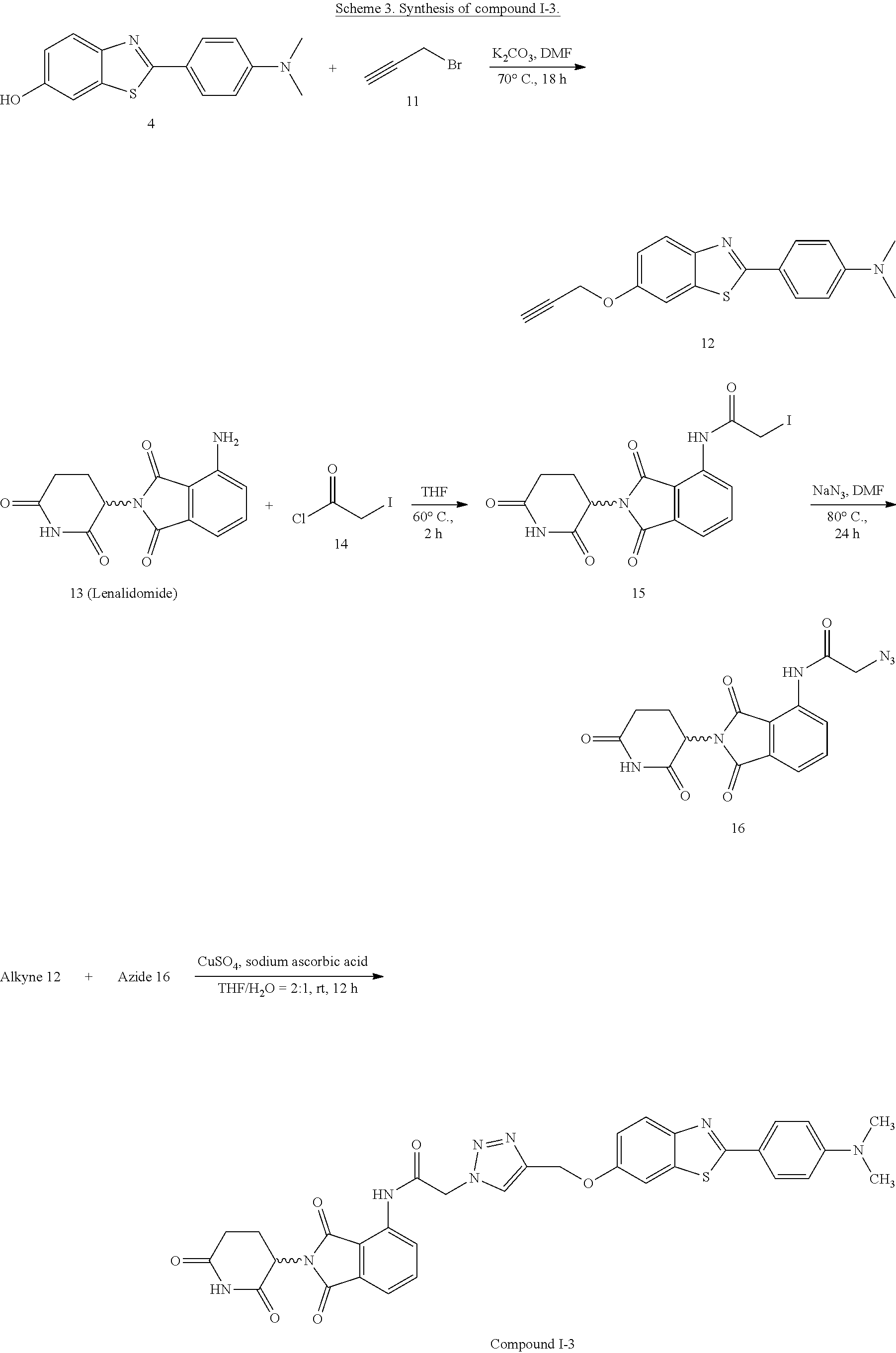

### Synthesis of N,N-dimethyl-4-(6-(prop-2-yn-1-yloxy)benzothiazol-2-yl)aniline (12)

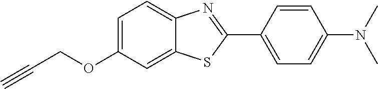 [SMILES: C#CCOc1ccc2nc(-c3ccc(N(C)C)cc3)sc2c1]

A mixture of 6-hydroxy substituted BTA compound 4 (100 mg, 0.37 mmol) and potassium carbonate (102 mg, 0.74 mmol) in DMF (2 mL) was stirred for 15 min. Propargyl bromide (56 μL, 0.74 mmol) was added, and the mixture was heated at 70° C. for 18 h. The reaction was quenched with 1 M HCl. The mixture was concentrated under reduced pressure. The organic phase was dried over MgSO₄, filtered, and concentrated under reduced pressure to give compound 12 (100 mg, 88% yield). C₁₈H₁₆N₂OS; pale yellow solid; UV-vis (DMSO) λₘₐₓ=362 nm (ε=17805 M⁻¹ cm⁻¹); ¹H NMR (400 MHz, CD₃OD) δ 7.89 (2H, d, J=9.2 Hz), 7.87 (1H, d, J=8.9 Hz), 7.40 (1H, d, J=2.1 Hz), 7.07 (1H, d, J=8.9 Hz), 6.74 (2H, d, J=9.2 Hz), 4.74 (2H, s), 3.02 (6H, s), 2.53 (1H, s); ¹³C NMR (100 MHz, CD₃Cl) δ 167.1, 154.9, 151.9, 135.5, 128.6, 126.4, 122.7, 121.4, 115.6, 111.7, 111.6, 106.1, 78.4, 75.8, 56.5, 40.2 (2×), 30.9; ESI-HRMS calcd for C₁₈H₁₆N₂OS: 307.0904, found m/z 307.0899 [M+H]⁺.

### Synthesis of N-(2-(2,6-dioxopiperidin-3-yl)-1-oxoisoindolin-4-yl)-2-iodoacetamide (15)

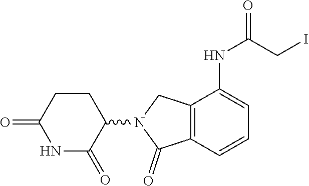 [SMILES: O=C1CCC(N2Cc3c(NC(=O)CI)cccc3C2=O)C(=O)N1]

Iodoacetyl chloride (34.3 μL, 0.386 mmol) was added dropwise to solution of lenalidomide (13) (100 mg, 0.39 mmol) in anhydrous THF (2 mL). The mixture was stirred at room temperature for 1 h, washed with saturated NaHCO_{3(aq)}, and triturated with Et₂O. The solids were collected by suction filtration, and rinsed with Et₂O to give compound 15 (155 mg, 94% yield). C₁₅H₁₄IN₃O₄; yellow solid; mp 284-286° C.; ¹H NMR (400 MHz, CD₃OD) δ 11.03 (1H, s), 10.20 (1H, s), 7.78 (1H, d, J=7.5 Hz), 7.53 (1H, t, J=7.5 Hz), 7.51 (1H, s), 5.15 (1H, dd, J=13.2, 5.2 Hz), 3.88 (2H, s), 4.41-4.28 (2H, m), 2.92-2.90 (1H, m), 2.48-2.43 (1H, m), 2.11-2.08 (2H, m); ¹³C NMR (100 MHz, DMSO-d₆) δ 172.9, 171.1, 168.4, 167.8, 133.8, 133.4, 132.7, 128.7, 125.3, 119.3, 51.5, 46.5, 40.1, 29.1, 22.7; ESI-HRMS calcd for C₁₅H₁₄IN₃O₄: 425.995, found m/z 425.992 [M+H]⁺.

### Synthesis of N-(2-(2,6-dioxopiperidin-3-yl)-1-oxoisoindolin-4-yl) 2-azido-acetamide (16)

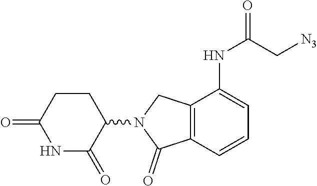 [SMILES: [N-]=[N+]=NCC(=O)Nc1cccc2c1CN(C1CCC(=O)NC1=O)C2=O]

A mixture of compound 15 (100 mg, 0.23 mmol) and sodium azide (30.4 mg, 0.47 mmol) in DMF (2 mL) was stirred at 80° C. for 24 h. The mixture was concentrated under reduced pressure and then extracted with EtOAc and H₂O. The organic phase was dried over MgSO₄, filtered, and concentrated under reduced pressure to give compound 16 (71 mg, 89% yield). C₁₅H₁₄N₆O₄; white solid; mp 172-174° C.; UV-vis (DMSO) λₘₐₓ=260 nm (ε=6219 M⁻¹ cm⁻¹); ¹H NMR (400 MHz, CD₃OD) δ 11.03 (1H, s), 10.09 (1H, s), 7.83 (1H, t, J=7.5 Hz), 7.11 (1H, d, J=7.5 Hz), 6.91 (1H, d, J=7.5 Hz), 5.15 (1H, dd, J=13.2, 5.2 Hz), 4.42-4.32 (2H, m), 4.10 (2H, s), 2.92-2.90 (1H, m), 2.80-2.79 (1H, m), 2.36-2.33 (1H, m), 2.04-2.01 (1H, m); ¹³C NMR (100 MHz, DMSO-d₆) δ 172.9, 171.1, 167.8, 166.7, 132.9, 132.8, 128.9, 125.5, 119.7, 116.9, 51.6, 51.1, 46.5, 31.3, 22.7; ESI-HRMS calcd for C₁₅H₁₄N₆O₄: 341.0998, found m/z 341.0997 [M+H]⁺.

### Synthesis of 2-(4-(((2-(4-(dimethylamino)phenyl)benzothiazol-6-yl)oxy)methyl)-1H-1,2,3-triazol-1-yl)acetamide (compound I-3)

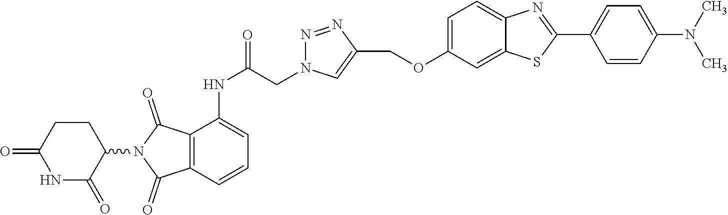 [SMILES: CN(C)c1ccc(-c2nc3ccc(OCc4cn(CC(=O)Nc5cccc6c5C(=O)N(C5CCC(=O)NC5=O)C6=O)nn4)cc3s2)cc1]

A mixture of alkyne compound 12 (50 mg, 0.16 mmol), azide compound 16 (50 mg, 0.15 mmol), sodium ascorbate (26 mg, 0.13 mmol) and CuSO₄ (7 mg, 0.044 mmol) in THF/H₂O (2:1) was stirred at room temperature for 12 h. The mixture was concentrated under reduced pressure, and then triturated with H₂O, hexane, EtOAc to give compound I-3 (84 mg, 88% yield). C₃₃H₃₀N₈O₅S; pale yellow solid; mp 223-238° C.; UV-vis (DMSO) λₘₐₓ=362 nm (ε=19824 M¹ cm⁻¹); ¹H NMR (400 MHz, DMSO-d₆) δ 11.03 (1H, s), 10.40 (1H, s), 8.30 (1H, s), 7.85 (1H, s), 7.84 (2H, d, J=9.2 Hz), 7.77 (1H, d, J=2.1 Hz), 7.55 (1H, t, J=7.5 Hz), 7.53 (1H, d, J=7.5 Hz), 6.80 (2H, d, J=7.5 Hz), 5.43 (2H, d, J=7.8 Hz), 5.25 (2H, d, J=8.2 Hz), 5.15 (1H, dd, J=13.2, 5.2 Hz), 4.38 (2H, s), 4.46-4.35 (2H, m), 3.01 (6H, s), 2.93-2.87 (1H, m), 2.76-2.74 (1H, m), 2.35-2.32 (1H, m), 2.08-2.03 (1H, m); ¹³C NMR (100 MHz, DMSO-d₆) δ 173.1, 171.2, 167.9, 165.8, 164.7, 155.6, 152.1, 148.6, 135.2, 133.8, 133.0, 132.9, 129.1 (2×), 128.5, 128.3, 126.7, 125.3, 122.6, 120.4, 119.8, 115.9, 111.9 (2×), 106.2, 72.3, 61.7, 60.4, 52.0, 51.7, 46.6, 31.3, 22.8; ESI-HRMS calcd for C₃₃H₃₀N₈O₅S: 649.1981, found m/z 649.1985 [M+H]⁺.

### 1.4 Compounds II-1a to II-1d

The compounds of formula II-1a to II-1d were synthesized in accordance with procedures described in Scheme 4. Briefly, the substitution reaction of 4-bromobenzyl bromide with triethoxyphosphine gave a phosphonate ester, which was treated with NaH and then reacted with 6-chloropyridine-3-carbaldehyde (also known as 6-chloronicotinaldehyde) to afford the styrylpyridine 19 in the (E)-configuration. The chlorine atom on the pyridine ring of 19 was then selectively substituted by an ethylene glycol linker (20a-20d) to give compounds 21a-21d, which underwent a copper catalyzed Ullmann amination with methylamine in a sealed tube to afford (N-methyl)arylamines 22a-22d. The amino group was protected as a tert-butoxycarbonyl (Boc) group, and the azido group was reduced by triphenylphosphine to give compounds 23a-23d. The coupling reaction between 23a-23d and the fluorine-substituted pomalidomide analog 8 was carried out, followed by removal of the Boc protecting group, to produce compounds II-1a, II-1b, II-1c and II-1d.

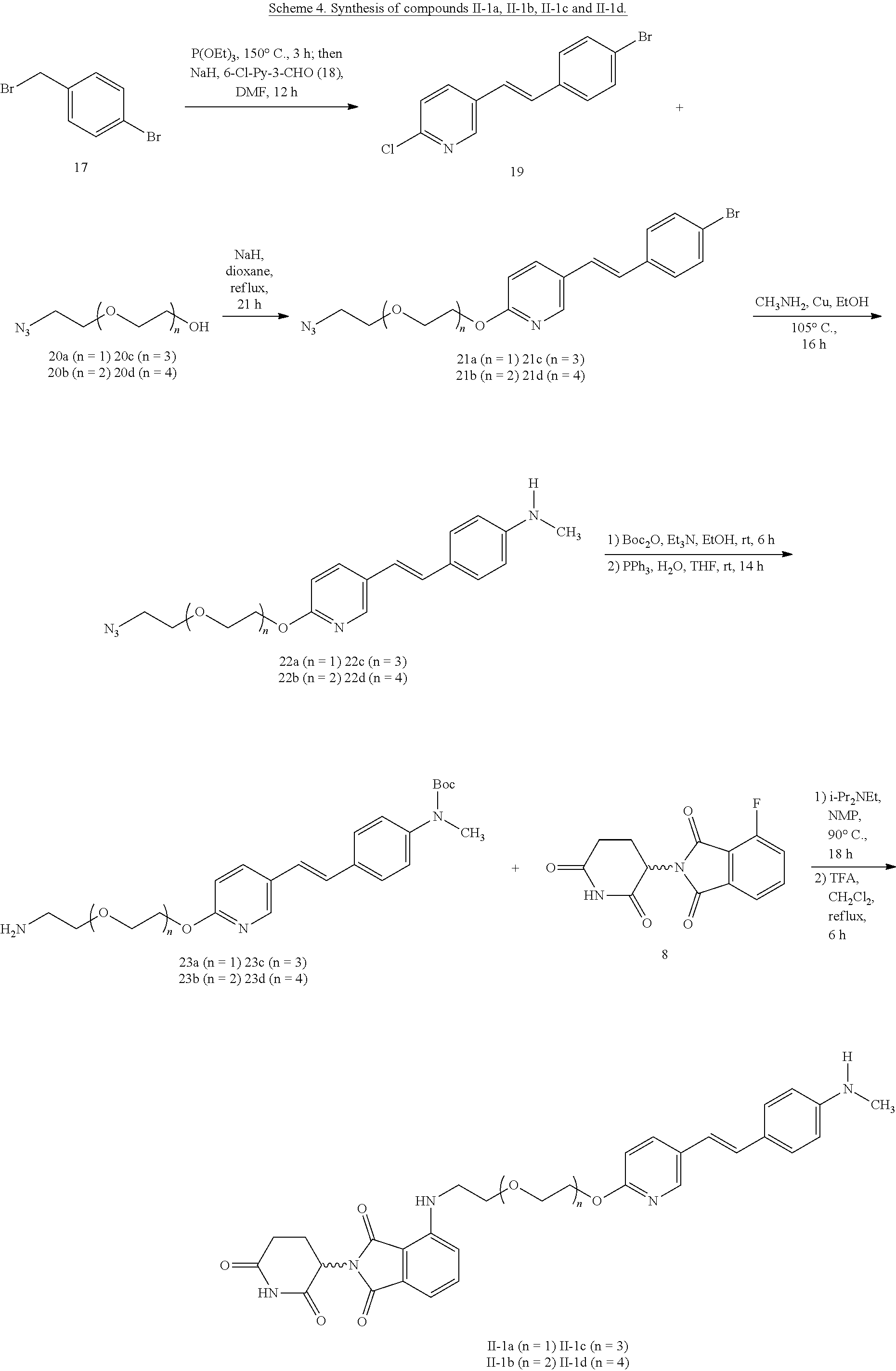

### Synthesis of (E)-2-(2-(2-(2-azidoethoxy)ethoxy)ethoxy)-5-(4-bromostyryl)pyridine (21b)

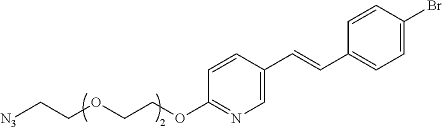

A solution of 2-(2-(2-azidoethoxy)ethoxy)ethanol (20b) (103 mg, 0.59 mmol) in dioxane (6 mL), was added NaH (47 mg, 1.18 mmol). The mixture was stirred for 1 h at room temperature, and (E)-5-(4-bromostyryl)-2-chloropyridine (19) (347 mg, 1.18 mmol) was added into the solution. The mixture was heated under reflux for 21 h, cooled, and extracted with EtOAc and H₂O. The combined organic phase was dried over MgSO₄, filtered, concentrated under reduced pressure and purified by flash chromatography on a silica gel column with elution of EtOAc/hexane (1:2) to give compound 21b (172 mg, 67% yield). C₁₉H₂₁BrN₄O₃; white solid; mp 48.0-49.0° C.; TLC (EtOAc/hexane=1:2) R_{f}=0.25; IR vₘₐₓ (neat) 2930, 2868, 2099, 1600, 1559, 1491, 1310, 1286, 1123, 1071, 958, 828 cm⁻¹; ¹H NMR (400 MHz, CDCl₃) δ 8.13 (s, 1H), 7.72 (dd, J=8.6, 2.2 Hz, 1H), 7.41 (d, J=8.4 Hz, 2H), 7.29 (d, J=8.4 Hz, 2H), 6.89 (q, J=16.4 Hz, 2H), 6.75 (d, J=8.6 Hz, 1H), 4.46 (t, J=4.7 Hz, 2H), 3.83 (t, J=4.7 Hz, 2H), 3.70-3.68 (m, 2H), 3.67-3.60 (m, 4H), 3.34 (t, J=5.0 Hz, 2H); ¹³C NMR (100 MHz, CDCl₃) δ 163.1, 145.7, 135.9, 135.3, 131.7 (2×), 127.7 (2×), 126.5, 126.2, 125.3, 121.2, 111.4, 70.6 (2×), 69.9, 69.6, 65.2, 50.5. ESI-HRMS calcd for C₁₉H₂₂BrN₄O₃: 433.0870, found: m/z 433.0879 [M+H]⁺.

### Synthesis of (E)-4-(2-(6-(2-(2-(2-azidoethoxy)ethoxy)ethoxy)pyridin-3-yl)vinyl)-N-methylaniline (22b)

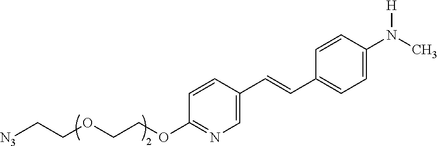

A mixture of compound 21b (508 mg, 1.18 mmol), copper (7.5 mg, 0.11 mmol) and 40% aqueous MeNH₂ solution (1 mL, 11 mmol) in EtOH (2 mL) was placed in an 8 mL screwed sealed tube. The mixture was stirred at 105° C. for 16 h, cooled, and extracted with EtOAc and H₂O. The combined organic phase was dried over MgSO₄, filtered, concentrated under reduced pressure and purified by flash chromatography on a silica gel column with elution of EtOAc/hexane (2:3) to give compound 22b (289 mg, 64% yield). C₂₀H₂₅N₅O₃; brown solid; mp 48.5-50.0° C.; TLC (EtOAc/hexane=2:3) R_{f}=0.38; IR vₘₐₓ (neat) 3396, 2920, 2849, 2105, 1608, 1525, 1489, 1308, 1283, 1181, 1126, 829 cm⁻¹; ¹H NMR (400 MHz, CDCl₃) δ 8.11 (d, J=2.3 Hz, 1H), 7.72 (dd, J=8.6, 2.3 Hz, 1H), 7.31 (d, J=8.6 Hz, 2H), 6.82 (q, J=16.2 Hz, 2H), 6.73 (d, J=8.6 Hz, 1H), 6.56 (d, J=8.6 Hz, 2H), 4.46 (t, J=4.7 Hz, 2H), 3.83 (t, J=4.7 Hz, 2H), 3.76-3.58 (m, 7H), 3.34 (t, J=5.1 Hz, 2H), 2.81 (s, 3H); ¹³C NMR (100 MHz, CDCl₃) δ 162.4, 148.7, 144.8, 135.0, 128.2, 127.5 (3×), 126.4, 120.2, 112.4 (2×), 111.1, 70.6 (2×), 69.9, 69.7, 65.1, 50.5, 30.6. ESI-HRMS calcd for C₂₀H₂₆N₅O₃: 384.2030, found: m/z 384.2047 [M+H]⁺.

### Synthesis of tert-butyl (E)-(4-(2-(6-(2-(2-(2-aminoethoxy)ethoxy)ethoxy)pyridin-3-yl)vinyl)phenyl)(methyl)carbamate (23b)

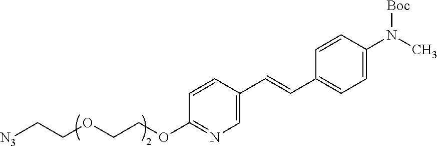

To a solution of amino compound 22b (166 mg, 0.43 mmol) and Et₃N (0.25 mL, 1.73 mmol) in EtOH (4 mL) was added di-tert-butyl dicarbonate (378 mg, 1.73 mmol). The mixture was stirred at room temperature for 6 h. The mixture was concentrated under reduce pressure and purified by flash chromatography on a silica gel column with elution of EtOAc/hexane (2:3) to the Boc-protecting compound (205 mg, 99% yield). The product (192 mg, 0.4 mmol) in THE (3 mL) was stirred with PPh₃ (311 mg, 1.19 mmol) and H₂O (21 μL, 1.19 mmol) at room temperature for 14 h. The mixture was concentrated under reduced pressure and purified by flash chromatography on a silica gel column with elution of CH₂Cl₂/MeOH (5:1) to give the amino compound 23b (173 mg, 95% yield). C₂₅H₃₅N₃O₅; yellow solid; mp 75.5-77.0° C.; TLC (CH₂Cl₂/MeOH=4:1) R_{f}=0.13; IR vₘₐₓ (neat) 3396, 2970, 2926, 1699, 1605, 1513, 1490, 1366, 1313, 1290, 1254, 1152, 1109 cm⁻¹; ¹H NMR (400 MHz, CDCl₃) δ 8.05 (d, J=2.4 Hz, 1H), 7.65 (dd, J=8.7, 2.4 Hz, 1H), 7.31 (d, J=8.4 Hz, 2H), 7.10 (d, J=8.4 Hz, 2H), 6.83 (d, J=1.7 Hz, 2H), 6.67 (d, J=8.7 Hz, 1H), 4.38 (t, J=4.7 Hz, 2H), 3.74 (t, J=4.7 Hz, 2H), 3.63-3.54 (m, 2H), 3.55-3.49 (m, 2H), 3.42 (t, J=5.1 Hz, 2H), 3.14 (s, 3H), 2.77 (s, 2H), 2.60 (br, 2H), 1.35 (s, 9H); ¹³C NMR (100 MHz, CDCl₃) δ 162.7, 154.3, 145.3, 142.8, 135.1, 133.8, 127.0, 126.4, 126.1 (2×), 125.1 (2×), 124.1, 111.0, 80.0, 72.3, 70.3, 69.9, 69.4, 64.9, 41.1, 36.8, 28.0 (3×). ESI-HRMS calcd for C₂₅H₃₆N₃O₅: 458.2649, found: m/z 458.2652 [M+H]⁺.

### Synthesis of (E)-2-(2,6-dioxopiperidin-3-yl)-4-((2-(2-(2-((5-(4-(methylamino)styryl)pyridin-2-yl)oxy)ethoxy)ethoxy)ethyl)amino)isoindoline-1,3-dione (Compound II-1b)

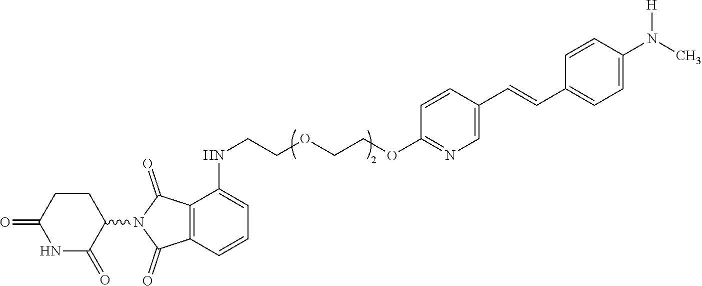

A mixture of fluorine-substituted pomalidomide analog 8 (664 mg, 2.4 mmol), compound 23b (846 mg, 1.85 mmol) and diisopropylethylamine (DIPEA) (660 μL, 3.7 mmol) in 1-methyl-2-pyrrolidone (NMP) (9 mL) was stirred at 90° C. for 22 h. The mixture was extracted with EtOAc and H₂O. The combined organic phase was dried over MgSO₄, filtered, concentrated under reduced pressure and purified by flash chromatography on a silica gel column with elution of EtOAc/CH₂Cl₂ (1:1) to give the coupling product (625 mg, 47% yield). A mixture of the product (200 mg, 0.28 mmol) and TFA (65 μL, 0.84 mmol) in CH₂Cl₂ (3 mL) was heated under reflux for 4 h. The reaction was quenched by addition of saturated NaHCO_{3(aq)}. The mixture was extracted with CH₂Cl₂ and H₂O. The combined organic phase was dried over MgSO₄, filtered, concentrated under reduced pressure, and then purified by flash chromatography on a silica gel column with elution of EtOAc/CH₂Cl₂ (2:1) to give the desired product II-1b (145 mg, 85% yield). The purity of compound II-1b was 98.3% as shown by HPLC on a silica column (Dikma, 10×250 mm, 10 m particle size), elution: EtOAc/hexane=4:1 at a flow rate of 3.0 mL/min, t_{R}=14.4 min. C₃₃H₃₅N₅O₇; orange solid; mp 98.5-100.0° C.; TLC (EtOAc/CH₂Cl₂=2:1) R_{f}=0.5; IR vₘₐₓ (neat) 3363, 3190, 2919, 2849, 1700, 1652, 1645, 1471, 1410, 1358 cm⁻¹; ¹H NMR (400 MHz, CDCl₃) δ 9.03 (s, 1H), 8.10 (s, 1H), 7.68 (d, J=8.6 Hz, 1H), 7.40 (t, J=7.4 Hz, 1H), 7.29 (d, J=7.7 Hz, 2H), 7.02 (d, J=7.0 Hz, 1H), 6.89-6.75 (m, 3H), 6.70 (d, J=8.6 Hz, 1H), 6.64 (d, J=7.7 Hz, 2H), 6.45 (s, 1H), 4.86 (q, J=5.7 Hz, 1H), 4.55-4.30 (m, 3H), 3.84 (t, J=4.4 Hz, 2H), 3.70-3.60 (m, 6H), 3.38 (d, J=4.6 Hz, 2H), 2.81 (s, 3H), 2.77-2.60 (m, 3H), 2.10-1.97 (m, 1H); ¹³C NMR (100 MHz, CDCl₃) δ 171.6, 169.1, 168.7, 167.4, 162.3, 148.3, 146.6, 144.8, 135.8, 134.9, 132.2, 128.0, 127.4 (2×), 127.3, 126.6, 120.2, 116.6, 112.6, 111.3 (2×), 111.0, 110.0, 70.5, 70.4, 69.6, 69.2, 65.1, 48.7, 42.1, 31.1, 30.6, 22.6. ESI-HRMS calcd for C₃₃H₃₆N₅O₇: 614.2609, found: m/z 614.2608 [M+H]⁺.

### Synthesis of (E)-2-((14-azido-3,6,9,12-tetraoxatetradecyl)oxy)-5-(4-bromostyryl)pyridine (21d)

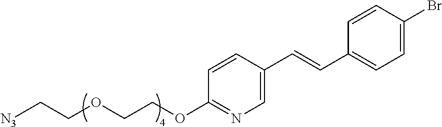

To a solution of compound 20d (306 mg, 1.16 mmol) in anhydrous dioxane (11 mL) was added NaH (93 mg, 2.33 mmol). The mixture was stirred for 30 min at room temperature, and a solution of compound 19 (686 mg, 2.33 mmol) in anhydrous DMF (1 mL) was added. The mixture was heated under reflux for 24 h, cooled, and extracted with EtOAc and H₂O. The combined organic phase was dried over MgSO₄, filtered, and concentrated under reduced pressure. The mixture was purified by silica gel chromatography (EtOAc/hexane=1:1) to obtain compound 21d (465 mg, 77% yield). C₂₃H₂₉BrN₄O₅; yellow syrup; TLC (EtOAc/hexane=1:1) R_{f}=0.4; IR vₘₐₓ (neat) 3050, 3022, 2870, 2103, 1600, 1493, 1391, 1312, 1071 cm⁻¹; ¹H NMR (400 MHz, CDCl₃) δ 8.15 (d, J=2.4 Hz, 1H), 7.76 (dd, J=8.8, 2.4 Hz, 1H), 7.44 (d, J=8.4 Hz, 2H), 7.32 (d, J=8.4 Hz, 2H), 6.98 (d, J=16.4 Hz, 1H), 6.88 (d, J=16.4 Hz, 1H), 6.77 (d, J=8.4 Hz, 1H), 4.47 (t, J=4.8 Hz, 2H), 3.84 (t, J=4.6 Hz, 2H), 3.71-3.63 (m, 14H), 3.36 (t, J=5.2 Hz, 2H); ¹³C NMR (100 MHz, CDCl₃) δ 163.2, 145.8, 136.0, 135.3, 131.7, 127.7, 126.6, 126.3, 125.4, 121.2, 111.5, 70.64, 70.61, 70.57, 70.55, 70.0, 69.7, 65.3, 50.6. ESI-HRMS calcd for C₂₃H₃₀BrN₄O₅: 521.1394, found: m/z 521.1376 [M+H]⁺.

### Synthesis of (E)-4-(2-(6-((14-azido-3,6,9,12-tetraoxatetradecyl)oxy)pyridin-3-yl)vinyl)-N-methylaniline (22d)

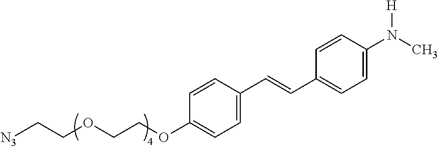

A mixture of compound 21d (450 mg, 0.863 mmol), copper (5.5 mg, 0.086 mmol) and 40% aqueous MeNH₂ solution (0.78 mL, 8.63 mmol) in EtOH (1.5 mL) was placed in an 8 mL screwed sealed tube. The mixture was stirred at 105° C. for 16 h, cooled, and extracted with EtOAc and H₂O. The combined organic phase was dried over MgSO₄, filtered, and concentrated under reduced pressure. The mixture was purified by silica gel chromatography (EtOAc/hexane=1:1 to 2:1) to obtain compound 22d (226 mg, 56% yield). C₂₄H₃₃N₅O₅; yellow syrup; TLC (EtOAc/hexane=1:1) R_{f}=0.38; IR vₘₐₓ (neat) 3511, 3386, 3017, 2875, 2814, 2104, 1607, 1525, 1488, 1283, 1125, 1054 cm⁻¹; ¹H NMR (400 MHz, CDCl₃) δ 8.09 (d, J=2.4 Hz, 1H), 7.71 (dd, J=9.2, 2.4 Hz, 1H), 7.29 (d, J=8.6 Hz, 2H), 6.86 (d, J=16.4 Hz, 1H), 6.78-6.71 (m, 2H), 6.54 (d, J=8.6 Hz, 2H), 4.44 (t, J=4.8 Hz, 2H), 3.81 (t, J=4.8 Hz, 2H), 3.69-3.60 (m, 14H), 3.33 (t, J=5.2 Hz, 2H), 2.80 (s, 3H); ¹³C NMR (100 MHz, CDCl₃) δ 162.4, 148.9, 144.8, 134.9, 128.1, 127.43, 127.41, 126.2, 120.1, 112.3, 111.1, 70.52, 70.5, 70.47, 70.45, 69.9, 69.6, 65.0, 50.5, 30.4. ESI-HRMS calcd for C₂₄H₃₄N₅O₅: 472.2554, found: m/z 472.2555 [M+H]⁺.

### Synthesis of tert-butyl (E)-(4-(2-(6-((14-amino-3,6,9,12-tetraoxatetradecyl)oxy)pyridin-3-yl)vinyl)phenyl)(methyl)carbamate (23d)

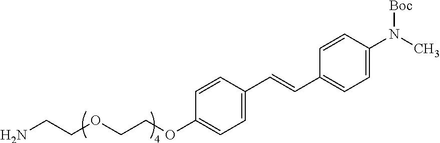

To a solution of amino compound 22d (204 mg, 0.43 mmol) and Et₃N (0.24 mL, 1.73 mmol) in EtOH (4.3 mL) was added di-tert-butyl dicarbonate (0.4 mL, 1.73 mmol). The mixture was stirred at room temperature for 10 h. The mixture was concentrated under reduce pressure and purified by silica gel chromatography (EtOAc/hexane=1:1) to obtain the Boc protecting compound Boc-22d (232 mg, 94% yield). A solution of Boc-22d (210 mg, 0.37 mmol) was stirred with PPh₃ (289 mg, 1.1 mmol) and H₂O (20 μL, 1.1 mmol) in THE (2.8 mL) at room temperature for 14 h. The mixture was concentrated under reduced pressure and purified by silica gel chromatography (CH₂Cl₂/MeOH=5:1) to obtain compound 23d (161 mg, 80% yield).

Compound Boc-22d: C₂₉H₄₁N₅O₇; yellow syrup; TLC (EtOAc/Hexane=1:1) R_{f}=0.44; IR vₘₐₓ (neat) 2979, 2931, 2871, 2103, 1697, 1604, 1565, 1512, 1490, 1354, 1311, 1289, 1151 cm⁻¹; ¹H NMR (400 MHz, CDCl₃) δ 8.15 (d, J=2.4 Hz, 1H), 7.76 (dd, J=8.8, 2.4 Hz, 1H), 7.41 (d, J=8.8 Hz, 2H), 7.19 (d, J=8.8 Hz, 2H), 6.93 (q, J=18.4, 16.4 Hz, 2H), 6.76 (d, J=8.8 Hz, 1H), 4.47-4.45 (m, 2H), 3.84-3.82 (m, 2H), 3.70-3.63 (m, 14H), 3.35 (t, J=5.2 Hz, 2H), 3.24 (s, 3H), 1.43 (s, 9H); ¹³C NMR (100 MHz, CDCl₃) δ 163.0, 154.6, 145.6, 143.1, 135.3, 134.1, 127.2, 126.7, 126.4, 125.4, 124.4, 111.4, 80.4, 70.64, 70.61, 70.58, 70.55, 70.0, 69.7, 65.2, 50.6, 37.1, 28.3. ESI-HRMS calcd for C₂₉H₄₂N₅O₇: 572.3079, found: m/z 572.3076 [M+H]⁺.

Compound 23d: C₂₉H₄₃N₃O₇; yellow syrup; TLC (CH₂Cl₂/MeOH=5:1) R_{f}=0.34; IR vₘₐₓ (neat) 3455, 3374, 3010, 2931, 2872, 1694, 1605, 1567, 1513, 1488, 1354, 1289, 1151 cm⁻¹; ¹H NMR (400 MHz, CDCl₃) δ 8.14 (d, J=2.4 Hz, 1H), 7.75 (dd, J=8.8, 2.4 Hz, 1H), 7.40 (d, J=8.8 Hz, 2H), 7.18 (d, J=8.8 Hz, 2H), 6.92 (dd, J=18.4, 16.4 Hz, 2H), 6.76 (d, J=8.8 Hz, 1H), 4.46-4.44 (m, 2H), 3.83-3.81 (m, 2H), 3.68-3.60 (m, 12H), 3.48 (t, J=5.2 Hz, 2H), 3.23 (s, 3H), 2.83 (br, 2H), 2.20 (br, 2H, —NH₂), 1.42 (s, 9H); ¹³C NMR (100 MHz, CDCl₃) δ 163.0, 154.6, 145.5, 143.1, 135.3, 134.0, 127.2, 126.7, 126.3, 125.4, 124.4, 111.3, 80.4, 72.9, 70.59, 70.52, 70.49, 70.47, 70.45, 70.2, 69.6, 65.2, 41.5, 37.1, 28.3. ESI-HRMS calcd for C₂₉H₄₄N₃O₇: 546.3174, found: m/z 546.3155 [M+H]⁺.

### Synthesis of (E)-2-(2,6-dioxopiperidin-3-yl)-4-((2-(2-(2-((5-(4-(methylamino)styryl)pyridin-2-yl)oxy)ethoxy)ethoxy)ethyl)amino)isoindoline-1,3-dione (compound II-1d)

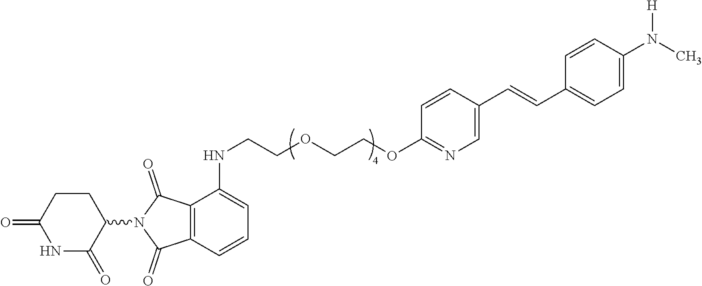

A mixture of compound 23d (306 mg, 0.56 mmol), compound 8 (119 mg, 0.73 mmol) and DIPEA (150 μL, 0.86 mmol) in NMP (2.5 mL) was stirred at 90° C. for 20 h. The solvent was concentrated under reduced pressure, and extracted with EtOAc and H₂O. The combined organic phase was dried over MgSO₄, filtered, and concentrated under reduced pressure. The mixture was purified by silica gel chromatography (EtOAc/CH₂Cl₂=1:1 containing 2% MeOH) to obtain the Boc protecting compound Boc-II-1d (215 mg, 48% yield).

To a solution of compound Boc-II-1d (200 mg, 0.28 mmol) in CH₂Cl₂ (1.7 mL) was added TFA (120 μL, 1.56 mmol). The mixture was heated under reflux for 6 h. The reaction was quenched by addition of saturated NaHCO_{3(aq)}. The mixture was extracted with CH₂Cl₂ and H₂O. The combined organic phase was dried over MgSO₄, filtered, and concentrated under reduced pressure. The mixture was purified by silica gel chromatography (EtOAc/CH₂Cl₂=1:1 containing 2% MeOH) to obtain compound II-1d (45.5 mg, 73% yield). The purity of compound II-1d was 98.9% as shown by HPLC on a Diamonsil column (Dikma, 10.0×250 mm, 5 m particle size), t_{R}=9.9 min (EtOAc/MeOH=9:1) at a flow rate of 2.0 mL/min.

Compound Boc-II-1d: C₄₂H₅₁N₅₀₁₁; yellow syrup; TLC (EtOAc/CH₂Cl₂=1:1) R_{f}=0.45; IR vₘₐₓ (neat) 3396, 2931, 2870, 1763, 1697, 1623, 1558, 1508, 1359, 1289, 1149, 1110 cm⁻¹; ¹H NMR (400 MHz, CDCl₃) δ 8.92 (br, 1H), 8.10 (d, J=2.8 Hz, 1H), 7.71 (dd, J=8.8, 2.8 Hz, 1H), 7.41-7.36 (m, 3H), 7.16 (d, J=8.8 Hz, 2H), 7.01 (d, J=6.8 Hz, 1H), 6.89 (s, 2H), 6.83 (d, J=8.4 Hz, 1H), 6.71 (d, J=8.8 Hz, 1H), 6.42 (t, J=5.6 Hz, 1H), 4.84 (dd, J=12.0, 5.2 Hz, 1H), 4.21 (t, J=4.8 Hz, 2H), 3.79 (t, J=4.8 Hz, 2H), 3.65-3.59 (m, 14H), 3.38 (q, J=4.1 Hz, 2H), 3.20 (s, 3H), 2.73-2.63 (m, 3H), 2.06-2.01 (m, 1H); ¹³C NMR (100 MHz, CDCl₃) δ 171.3, 169.1, 168.5, 167.5, 162.9, 154.4, 146.6, 145.4, 142.9, 135.8, 135.2, 133.9, 132.3, 127.1, 126.5, 126.2, 125.3, 124.3, 116.6, 111.3, 111.2, 110.1, 80.2, 70.50, 70.48, 70.43, 70.10, 70.36, 69.5, 69.3, 65.1, 48.7, 42.2, 37.0, 31.2, 28.2, 22.6. ESI-HRMS calcd for C₄₂H₅₂N₅O₁₁: 802.3658, found: m/z 802.3627 [M+H]⁺.

Compound II-1d: C₃₇H₄₃N₅O₉; yellow syrup; TLC (EtOAc/CH₂Cl₂=1:1) R_{f}=0.25; IR vₘₐₓ (neat) 3396, 2963, 2923, 2871, 1719, 1697, 1607, 1521, 1506, 1361, 1322, 1259, 1180, 1111 cm⁻¹; ¹H NMR (400 MHz, CDCl₃) δ 8.43 (br, 1H), 8.10 (d, J=2.0 Hz, 1H), 7.72 (dd, J=8.4, 2.4 Hz, 1H), 7.44 (dd, J=8.4, 7.2 Hz, 1H), 7.31 (d, J=8.8 Hz, 2H), 7.06 (d, J=7.2 Hz, 1H), 6.89-6.85 (m, 2H), 6.79 (s, 1H), 6.75-6.71 (m, 1H), 6.56 (d, J=8.4 Hz, 2H), 6.45 (t, J=5.6 Hz, 1H), 4.86 (dd, J=12.4, 5.6 Hz, 1H), 4.45 (t, J=4.8 Hz, 2H), 3.83 (t, J=4.8 Hz, 2H), 3.68-3.63 (m, 14H), 3.42 (q, J=4.1 Hz, 2H), 2.83 (s, 3H), 2.78-2.65 (m, 3H), 2.10-2.04 (m, 1H); ¹³C NMR (100 MHz, CDCl₃) δ 171.1, 169.2, 168.4, 167.6, 162.4, 149.0, 146.8, 144.9, 136.0, 135.0, 132.4, 128.3, 128.2, 127.5, 126.4, 120.2, 116.8, 112.4, 112.6, 111.2, 110.2, 70.68, 70.63, 70.59, 70.57, 70.55, 70.51, 69.7, 69.4, 65.1, 48.8, 42.3, 31.3, 30.6, 22.7. ESI-HRMS calcd for C₃₇H₄₃N₅NaO₉: 724.2953, found: m/z 724.2984 [M+Na]⁺.

### 1.5 Compounds II-2a to II-2d

The compounds II-2a, II-2b, II-2c and II-2d independently having the (pyridylvinyl)aniline and pomalidomide moieties connected by an aliphatic chain were synthesized in accordance with procedures described in Scheme 5.

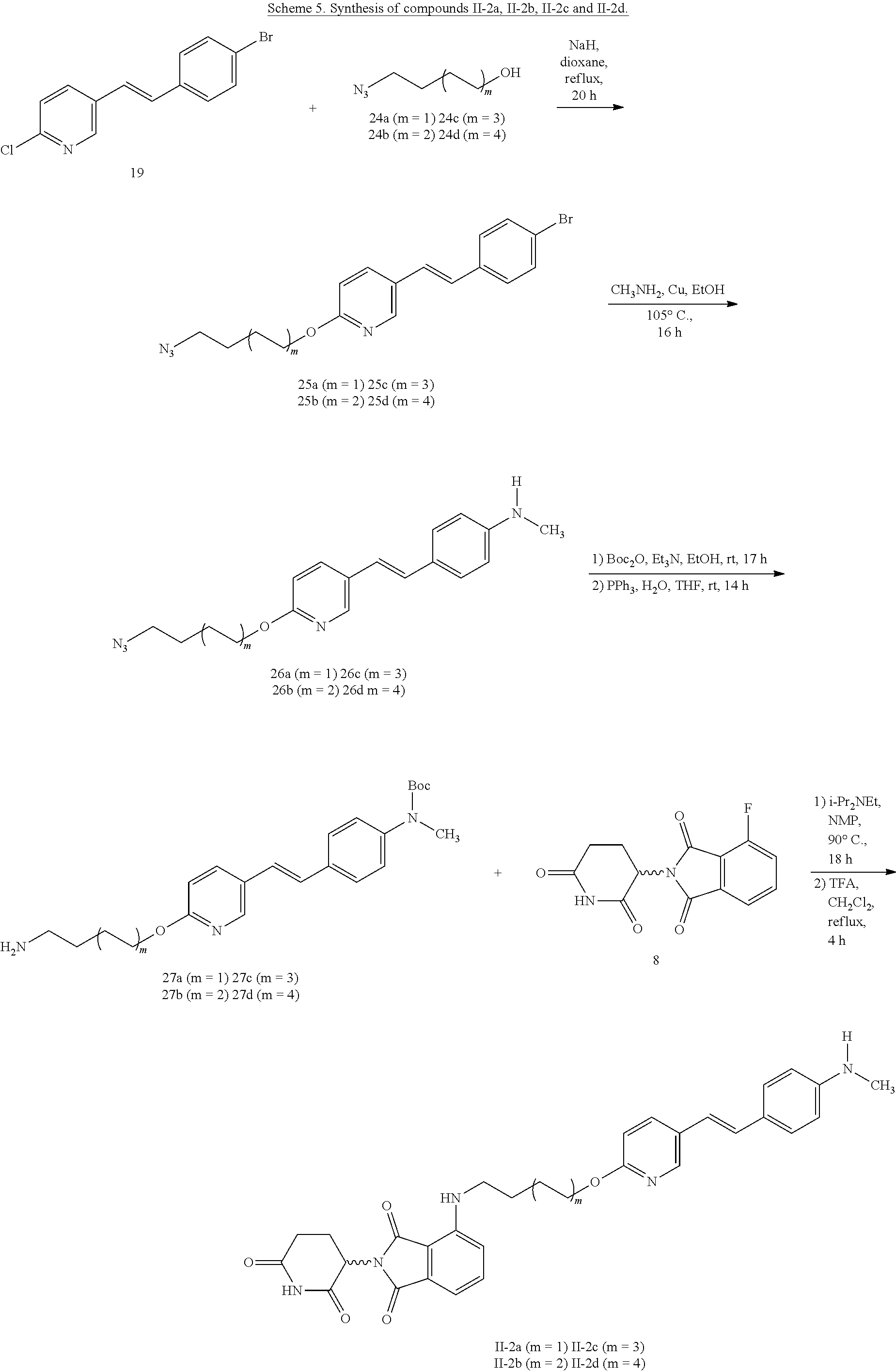

### Synthesis of (E)-2-((10-azidodecyl)oxy)-5-(4-bromostyryl)pyridine (25d)

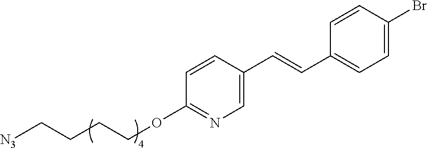

A solution of 10-bromodecan-1-ol (2 mL, 10 mmol) and NaN₃ (720.5 mg, 11 mmol) in acetone/water (30 mL, 2:1, v/v) was stirred and heated under reflux for 18 h. The mixture was concentrated under reduced pressure and extracted with Et₂O and H₂O. The organic phase was collected, dried over MgSO₄, filtered and concentrated under reduced pressure. The residue was purified by silica gel chromatography (EtOAc/hexane=0:100 to 20:80) to obtain 10-azidodecanol (1.77 g, 89% yield).

A solution of the above-prepared 10-azidodecanol compound (398 mg, 2 mmol) and NaH (200 mg, 5 mmol, 60% in mineral oil) in anhydrous dioxane (15 mL) was stirred for 1 h at room temperature, then (E)-5-(4-bromostyryl)-2-chloropyridine (1.18 g, 4 mmol) was added. The mixture was heated under reflux for 20 h, cooled, and extracted with EtOAc and H₂O. The combined organic phase was dried over MgSO₄, filtered, and concentrated under reduced pressure. The residue was purified by silica gel chromatography (EtOAc/hexane=0:100 to 20:80) to obtain compound 25d (650 mg, 71% yield). C₂₃H₂₉BrN₄O; white solid; ¹H NMR (400 MHz, CDCl₃) δ 8.17 (d, J=2.5 Hz, 1H), 7.75 (dd, J=8.7, 2.5 Hz, 1H), 7.44 (d, J=8.5 Hz, 2H), 7.32 (d, J=8.5 Hz, 2H), 6.99 (d, J=16.4 Hz, 1H), 6.87 (d, J=16.3 Hz, 1H), 6.71 (d, J=8.6 Hz, 1H), 4.28 (t, J=6.7 Hz, 2H), 3.23 (t, J=7.0 Hz, 3H), 1.81-1.70 (m, 2H), 1.57 (quin, J=7.0 Hz, 4H), 1.42 (td, J=9.2, 4.6 Hz, 2H), 1.38-1.21 (m, 16H); ¹³C NMR (100 MHz, CDCl₃) δ 163.7, 146.0, 136.1, 135.3, 131.8 (2×), 127.8 (2×), 126.4, 126.0, 125.6, 121.2, 111.3, 66.3, 51.5, 29.4 (3×), 29.1 (2×), 28.8, 26.7, 26.0.

### Synthesis of (E)-4-(2-(6-((10-azidodecyl)oxy)pyridin-3-yl)vinyl)-N-methylaniline (26d)

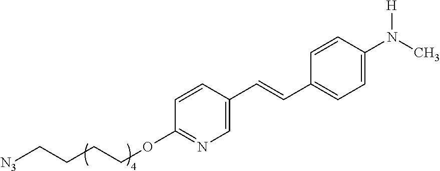

A mixture of compound 25d (229 mg, 0.5 mmol), copper (3.2 mg, 0.05 mmol) and 40% aqueous MeNH₂ solution (0.9 mL, 1 mmol) in EtOH (2 mL) was placed in an 8 mL screwed sealed tube. The mixture was stirred and heated at 105° C. for 16 h, cooled, and extracted with CH₂Cl₂ and H₂O. The combined organic phase was dried over MgSO₄, filtered, and concentrated under reduced pressure. The residue was purified by silica gel chromatography (EtOAc/hexane=0:100 to 10:90) to obtain compound 26d (94 mg, 46% yield). C₂₄H₃₃N₅O; white solid; ¹H NMR (400 MHz, CDCl₃) δ 8.14 (d, J=2.5 Hz, 1H), 7.73 (dd, J=8.7, 2.5 Hz, 1H), 7.36-7.29 (m, 2H), 6.86 (t, J=17.4 Hz, 2H), 6.69 (d, J=8.6 Hz, 1H), 6.60 (d, J=8.1 Hz, 2H), 4.26 (t, J=6.7 Hz, 2H), 3.23 (t, J=7.0 Hz, 2H), 2.85 (s, 3H), 1.75 (q, J=7.0 Hz, 2H), 1.57 (quin, J=7.1 Hz, 2H), 1.48-1.38 (m, 2H), 1.37-1.28 (m, 10H); ¹³C NMR (100 MHz, CDCl₃) δ 163.0, 148.7, 145.2, 135.0, 128.0, 127.5, 127.2, 120.6, 112.7, 111.0, 66.2, 51.5, 30.8, 29.4, 29.4, 29.3, 29.1, 29.1, 28.8, 26.7, 26.0.

### Synthesis of tert-butyl (E)-(4-(2-(6-((10-azidodecyl)oxy)pyridin-3-yl)vinyl)phenyl)(methyl)carbamate (27d)

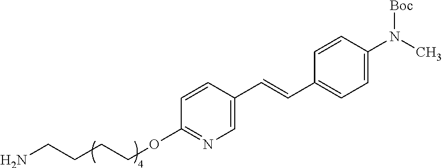

A solution of compound 26d (53 mg, 0.13 mmol), di-tert-butyl dicarbonate (0.12 mL, 0.52 mmol) and Et₃N (0.072 mL, 0.52 mmol) in EtOH (1.3 mL) was stirred at room temperature for 17 h. The mixture was concentrated under reduce pressure, and purified by silica gel chromatography (EtOAc/hexane=0:100 to 10:90) to obtain the Boc protecting derivative Boc-26d (67 mg, 94% yield). C₂₉H₄₁N₅O₃; white solid; ¹H NMR (400 MHz, CDCl₃) δ 8.17 (d, J=2.4 Hz, 1H), 7.76 (dd, J=8.7, 2.4 Hz, 1H), 7.45-7.37 (m, 2H), 7.20 (d, J=8.3 Hz, 2H), 6.94 (d, J=3.8 Hz, 2H), 6.71 (d, J=8.6 Hz, 1H), 4.27 (t, J=6.7 Hz, 2H), 3.24 (d, J=7.0 Hz, 5H), 1.75 (q, J=6.9 Hz, 3H), 1.57 (quin, J=7.0 Hz, 2H), 1.44 (s, 10H), 1.38-1.26 (m, 11H); ¹³C NMR (100 MHz, CDCl₃) δ 163.5, 154.6, 145.8, 143.1, 135.2, 134.2, 127.1, 126.4, 125.5, 124.6, 111.2, 80.4, 66.3, 51.5, 37.2, 29.4, 29.4, 29.3, 29.1, 29.0, 28.8, 28.3, 26.7, 26.0.

The above-prepared compound Boc-26d (51 mg, 0.1 mmol) was stirred with PPh₃ (79 mg, 0.3 mmol) and H₂O (5.4 μL, 0.3 mmol) in THE (1 mL) at room temperature for 14 h. The mixture was concentrated under reduced pressure and purified by silica gel chromatography (CH₂Cl₂/MeOH=100:0 to 95:5) to obtain compound 27d (41 mg, 85% yield). C₂₉H₄₃N₃O₃; white solid; ¹H NMR (400 MHz, CDCl₃) δ 8.16 (d, J=2.4 Hz, 1H), 7.75 (dd, J=8.7, 2.5 Hz, 1H), 7.41 (d, J=8.5 Hz, 2H), 7.19 (d, J=8.3 Hz, 2H), 6.93 (d, J=4.0 Hz, 2H), 4.26 (t, J=6.7 Hz, 2H), 3.24 (s, 3H), 1.74 (quin, J=6.9 Hz, 2H), 1.52 (dt, J=12.3, 6.0 Hz, 2H), 1.41 (s, 11H), 1.29 (d, J=16.1 Hz, 10H); ¹³C NMR (100 MHz, CDCl₃) δ 163.6, 154.6, 145.8, 143.1, 135.2, 134.2, 127.0, 126.4, 125.5, 124.6, 111.1, 80.4, 66.3, 37.2, 29.5, 29.3, 29.3, 29.0, 28.3, 26.8, 26.0.

### Synthesis of (E)-2-(2,6-dioxopiperidin-3-yl)-4-((10-((5-(4-(methylamino)styryl)pyridin-2-yl)oxy)decyl)amino)isoindoline-1,3-dione (II-2d)

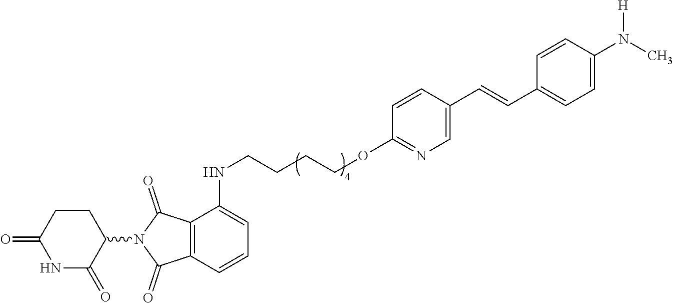

A mixture of compound 27d (94 mg, 0.195 mmol), compound 8 (41 mg, 0.15 mmol) and DIPEA (52 μL, 0.3 mmol) in NMP (1 mL) was stirred at 90° C. for 18 h. The mixture was concentrated under reduced pressure, and extracted with EtOAc and H₂O. The combined organic phase was dried over MgSO₄, filtered, and concentrated under reduced pressure. The residue was purified by silica gel chromatography (EtOAc/CH₂Cl₂=0:100 to 10:90) to obtain the Boc protecting derivative Boc-II-2d.

C₄₂H₅₁N₅O₇; yellow syrup; ¹H NMR (400 MHz, CDCl₃) δ 8.18 (s, 1H), 8.05 (s, 1H), 7.77 (dd, J=8.5, 2.3 Hz, 1H), 7.51-7.38 (m, 4H), 7.20 (d, J=8.3 Hz, 2H), 7.06 (d, J=7.0 Hz, 1H), 6.94 (d, J=3.2 Hz, 2H), 6.86 (d, J=8.5 Hz, 1H), 6.72 (d, J=8.6 Hz, 1H), 6.20 (d, J=6.1 Hz, 1H), 4.93-4.84 (m, 1H), 4.28 (t, J=6.7 Hz, 2H), 3.25 (s, 3H), 2.92-2.64 (m, 3H), 2.15-2.07 (m, 1H), 1.76 (quin, J=7.0 Hz, 3H), 1.67-1.49 (m, 19H), 1.43 (d, J=5.0 Hz, 17H), 1.36-1.19 (m, 22H).

The above-prepared compound Boc-II-2d (10 mg, 0.014 mmol) in CH₂Cl₂ (0.5 mL) was stirred with TFA (10 μL, 0.13 mmol) at 30° C. for 4 h. The mixture was concentrated under reduced pressure to obtain compound II-2d (6.8 mg, 78% yield).

C₃₇H₄₃N₅O₉; yellow syrup; ¹H NMR (400 MHz, CDCl₃) δ 8.30 (s, 1H), 7.93 (d, J=8.6 Hz, 1H), 7.50-7.42 (m, 4H), 7.24 (d, J=12.9 Hz, 8H), 7.08-7.03 (m, 2H), 6.95 (s, 2H), 6.86 (d, J=8.6 Hz, 3H), 4.89 (dd, J=12.0, 5.3 Hz, 2H), 4.45 (s, 3H), 4.34-4.20 (m, 7H), 3.23 (t, J=7.0 Hz, 3H), 2.98 (s, 4H), 2.90-2.64 (m, 6H), 2.15-2.05 (m, 2H), 1.82-1.74 (m, 4H), 1.64 (dq, J=14.8, 6.8 Hz, 4H), 1.39 (d, J=7.5 Hz, 5H), 1.30 (d, J=5.0 Hz, 19H), 1.26 (s, 8H), 1.23 (s, 6H), 0.90-0.80 (m, 5H). ESI-HRMS calcd for C₃₇H₄₄N₅O₅: 638.3337, found: m/z 638.3359 [M+H]⁺.

### Example 2 the Present Compounds Degraded Pathological Protein Aggregates

### 2.1 Degradation of C-TDP-43 Aggregates

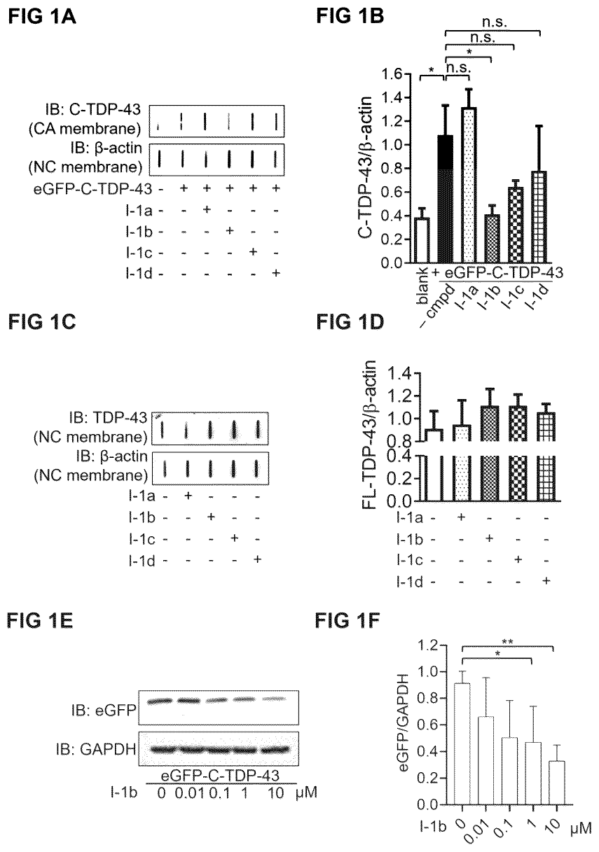

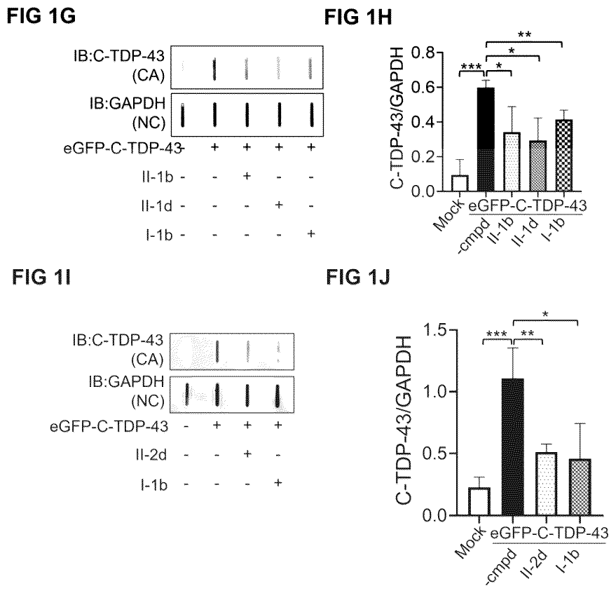

In this example, the effect of the present compound on C-TDP-43 aggregates was investigated. To this purpose, the eGFP-TDP-43₂₀₈₋₄₁₄ (hereafter referred as eGFP-C-TDP-43) expressed Neuro-2a cells were first treated with tested compound (e.g., any one of the compounds of formula (I) or (II), then, cells were lysed and the insoluble eGFP-C-TDP43 aggregates were quantified by use of filter trap assay as described in the “Materials and Methods” section; for comparison, slot blot assay was also performed by using the nitrocellulose (NC) membrane for the loading control. Results are depicted in FIG. 1.

Based on the amount of j-actin (internal control) on NC membrane, overexpression of eGFP-C-TDP-43 did induce a significant amount of C-TDP-43 species (mock control, 1.08±0.31, the black bar in FIG. 1, (B)) comparing with that in blank control of Neuro-2a cells (0.38±0.08, the white bar in FIG. 1, (B)). Interestingly, treatment with compound I-1b markedly reduced the insoluble eGFP-C-TDP-43 (0.41±0.06 in FIG. 1, (B)) than that of the mock control and other treated groups (0.64-1.31) (FIG. 1, (A) and (B)). In comparison, it was further demonstrated that compound I-1b (1.11±0.14) and other treated groups did not significantly affect the level of endogenous TDP-43 protein (FL-TDP-43) comparing to that of mock control (0.91±0.07) (FIG. 1, (C) and (D)). As compound I-1b outperformed other compounds (i.e., compounds I-1a, I-1c, and I-1d) in terms of eGFP-C-TDP-43 removal, SDS-PAGE was performed to verify the dose dependency of compound I-1b on the degradation of eGFP-C-TDP-43. It was confirmed that compound I-1b degraded eGFP-C-TDP-43 in a dose-dependent manner (FIG. 1, (E) and (F)).

As to the effect of compounds of formula (II), it was found that compounds II-1b, II-1d, and II-2d at the dose of 5 μM could significantly reduce the level of C-TDP-43 aggregate as compared to that of the control group (FIG. 1, (G), (H), (I) and (J)).

Taken together, the data in this example collectively indicated that the compounds of formula (I) or (II) facilitated in the degradation of C-TDP-43 aggregates.

### 2.2 Cell Viability in C-TDP-43 Expressed Neuro-2a Cells

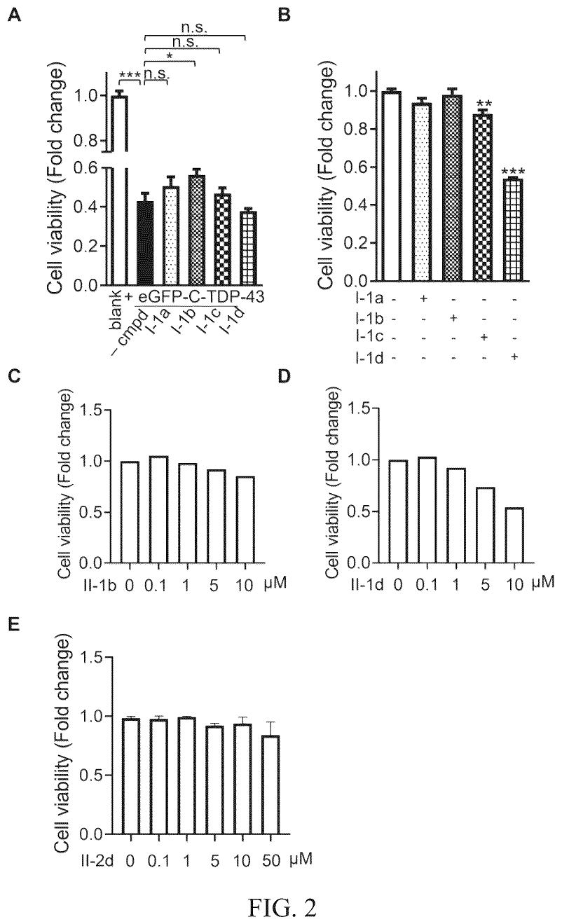

In this example, whether the compounds of formula (I) or (II) could relieve C-TDP-43-mediated cytotoxicity was examined. Results are provided in FIG. 2.

It was found that overexpression of eGFP-C-TDP-43 resulted in a decrease in cell viability (0.43±0.04 vs 1±0.03 (blank control). Notably, treatment with compound I-1b significantly enhanced cell viability (0.56±0.04) as compared to the mock group and other treated groups (0.37-0.50) (FIG. 2, (A)). Further, compounds I-1a and I-1b exhibited neglectable cytotoxicity at 5 μM concentration comparing to mock group of Neuro-2a cells (FIG. 2, (B)). In parallel, compound II-1b with shorter linker (FIG. 2, (C)) also exhibited less cytotoxicity at 5 or 10 μM concentration as compared to that of compound II-1d (FIG. 2, (D)). In addition, it was also found that compound II-2d (i.e., the compound with (CH₂CH₂)ₘ alkyl linker) exhibited lower toxicity than compound II-1d (i.e., the compound with (OCH₂CH₂)ₙ ethylene glycol linker) (FIG. 2, (E)).

### 2.3 Degradation of PolyQ-Expanded Huntingtin Aggregates

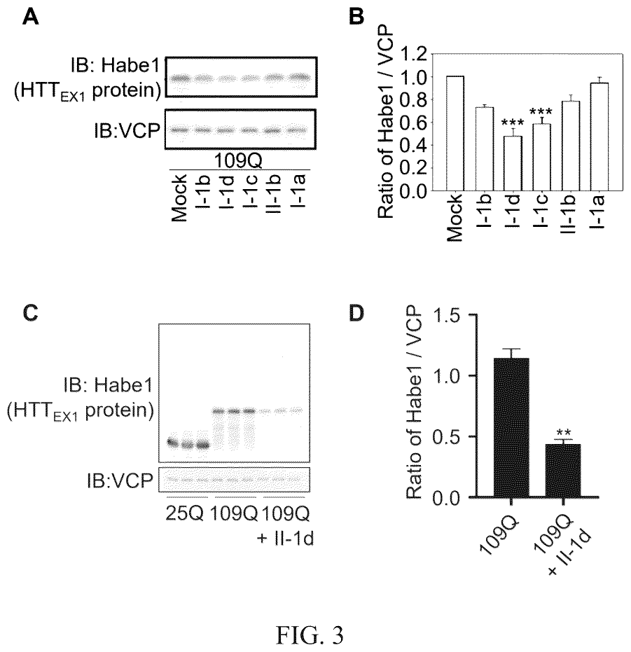

In this example, the effect of the present compound on huntingtin (HTT) protein (a protein known to cause Huntington's disease) was investigated. To this purpose, Neuro-2a cells were transiently transfected with pcDNA3-109Q-HTT_{EX1} (i.e., the polyQ-expanded N terminus of HTT translated from the exon 1 of human huntingtin gene (designated 109Q-HTT_{EX1})) and then exposed to the indicated compound (5 μM) for additional 24 h. Total protein lysates were harvested for Western blot analyses, and results are depicted in FIG. 3.

According to data presented in FIG. 3, compounds I-1c and I-1d, and compound II-1d could independently reduce the expression of 109Q-HTT_{EX1} by approximately 50%, while compound II-1b resulted in only 20% reduction of 109Q-HTT_{EX1}.

### 2.4 Degradation of Pathogenic Tau Protein

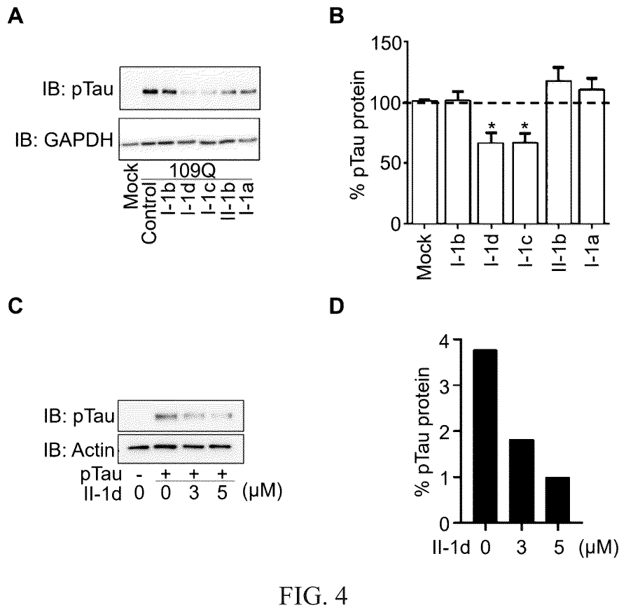

Hyperphosphorylated tau protein is a major hallmark of Alzheimer's disease and many other neurological disorders (including Huntington's disease). To investigate the effects of the compounds of formula (I) or formula (II) on the pathogenic tau protein (designated pTau), SH-SY5Y-Tau-P301L cells were treated with doxycycline (1 μg/mL) (which induced the expression of pathogenic tau protein (pTau), and the indicated compound (5 μM) for 24 h. Total protein lysates were then harvested and analyzed by Western blot analyses. Results are illustrated in FIG. 4.

It was found that compounds I-1c, I-1d, and II-1d independently reduced the expressed level of pTau by approximately 40%, while compound IT-1b exerted negligible effect on the degradation of pTau protein.

### Example 3 the Present Compounds Degraded Pathological Protein Aggregates in Nematode C. elegans

In this example, a model organism nematode C. elegans was used to investigate the effect of the present compounds of formula (I) or (II). Nematode C. elegans has advantages including short lifespan, optical transparency and quantifiable locomotion, makes it an ideal system for phenotypical scoring in drug discovery. Since YFP—C-TDP-43 C. elegans develops pathogenic protein aggregates and exhibits severe locomotive defects, it is used as a model organism for examining the effects of a candidate compound in alleviating C-TDP-43 neurotoxicity.

A transgenic C. elegans strain YFP—C-TDP-43₂₁₉₋₄₁₄, which ectopically expressed 25 kDa C-terminal fragment of human TDP-43 fused with YFP in nervous system, was used to evaluate whether a test compound possessed any therapeutic effect against pathogenic protein aggregates. Accordingly, accumulated aggregates and swimming pattern of YFP—C-TDP-43 C. elegans in response to the treatment of compound I-1b were examined.

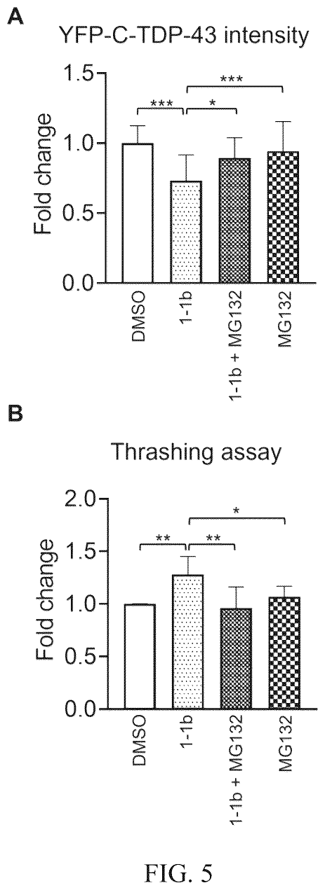

As YFP—C-TDP-43 readily forms cytosolic aggregates in nervous system, the YFP—C-TDP-43 aggregates both in the neuron processes and neuron bodies of the ventral cord were monitored. Compared to the DMSO control group or MG132-treated group, C. elegans treated with compound I-1b (5 μM) had fewer aggregates with reduced fluorescence intensity, quantified results are depicted in FIG. 5, (A). In comparison, YFP—C-TDP-43 aggregates persisted in the case when C. elegans were treated with the combination of compound I-1b and MG132 (a proteosome inhibitor), supporting that compound I-1b reduced aggregates via a proteasome-dependent pathway. The effect of compound I-1b on the locomotive defects of YFP—C-TDP-43 C. elegans was also examined. C. elegans swimming (also known as thrashing) in buffer was taken as the phenotypical assessment for the drug efficacy. As the DMSO control and MG132-treated groups of C. elegans exhibited slow bending frequency, the C. elegans treated with compound I-1b exhibited higher bending frequency (28% increases) as compared to that of the DMSO control group (FIG. 5, (B)). Notably, the combined treatment with compound I-1b and MG132 rendered a decreased bending frequency of C. elegans by 30% as compared to that treated with compound I-1b alone.

Taken together, the data clearly demonstrated the beneficial effect of compound I-1b in reducing C-TDP-43 aggregates and improving the motility of the transgenic C. elegans.

### Example 4 the Present Compounds Decreased Compactness and Population of C-TDP-43 Oligomers in Cells

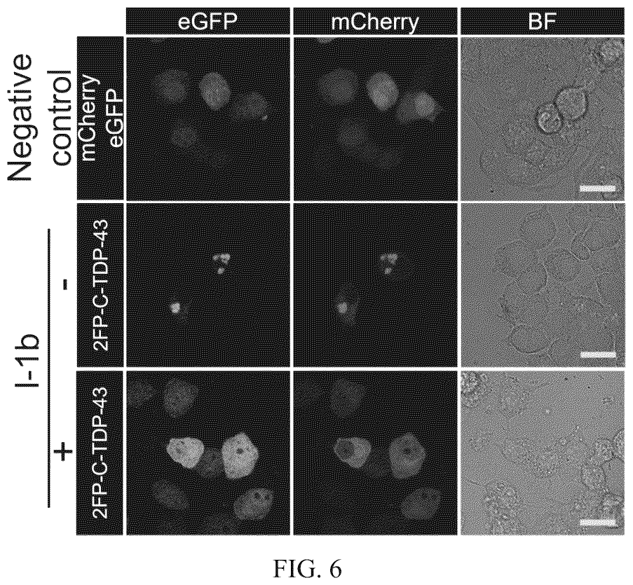

Recent studies had speculated that TDP-43 oligomers might play a role in ALS-related animals (Asakawa, K. et al. Nat. Commun. 2020, 11 (1), 1004) and frontotemporal lobar degeneration (FTLD) patients (Fang, Y. S. et al. Nat. Commun. 2014, 5, 4824). As compound I-1b could reduce TDP-43 aggregates, its possible interference with TDP-43 oligomers was further investigated in this example. To address this issue, two fluorescence protein labelled C-TDP-43 cell model (2FP—C-TDP-43) was generated by co-expressing eGFP-C-TDP-43 (FRET donor) and mCherry-C-TDP-43 (FRET acceptor) in Neuro-2a cells in accordance with procedures described in the “Materials and Methods” section, images of the Neuro-2a cells treated with or without Compound I-1b were then taken and analyzed. It was found that in Neuro-2a cells, both donor and acceptor shared similar expressivity and aggregation-prone properties (FIG. 6).

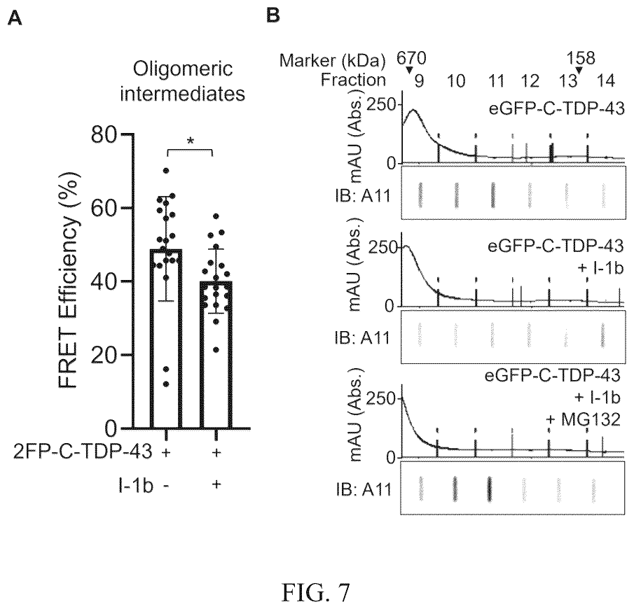

To assess C-TDP-43 oligomeric intermediates, fluorescence lifetime imaging microscopy (FLIM) was employed to measure the efficiency of Forster resonance energy transfer (E_{FRET}) of the fluorescence protein-tagged C-TDP-43 in the presence or absence of compound I-1b. The FLIM-FRET technique enables the collection of photon counts of the donor eGFP from the Neuro-2a cells co-expressed with eGFP+mCherry (negative control) or co-expressed with eGFP-C-TDP-43+mCherry-C-TDP-43 (2FP—C-TDP-43) in the absence or presence of compound I-1b after 48 h incubation. By setting the threshold of the photon counts, the soluble C-TDP-43 could be specifically analyzed. The lifetime of C-TDP-43 oligomeric intermediates was obtained through 2-component fitting from the highlighted soluble regions (data not shown). The fitted lifetime results are converted into E_{FRET} map (data not shown) and histogram (data not shown), which showed the averaged E_{FRET} values of 3.36%, 28.12% and 22.4% for the negative control (eGFP+mCherry) and expressing 2FP—C-TDP-43 in the absence and presence of compound I-1b, respectively. The high E_{FRET} (28.12%) in cells expressing 2FP—C-TDP-43+ might be attributable to the enriched C-TDP-43 oligomeric intermediates in the cytosol, whereas the presence of compound I-1b might suppress the oligomeric intermediates to lower the E_{FRET} (22.4%). Statistically by applying ISS software (VistaVision), it was found that addition of compound I-1b decreased the E_{FRET} of the C-TDP-43 oligomeric intermediates from 48.92% to 40.11% (FIG. 7, (A)).

To double check the interference of compound I-1b with the C-TDP-43 oligomeric intermediates, size-exclusion chromatography (SEC) and oligomer-specific antibody (All) were used to analyze the lysate of the Neuro-2a cells overexpressed eGFP-C-TDP-43 in the presence or absence of compound I-1b. Each fraction of the lysate was subjected to slot blot assay with oligomer-specific antibody A11 (FIG. 7, (B)). Comparing to the added markers (670 and 158 kDa), eGFP-C-TDP-43 oligomeric intermediates mostly occurred in fractions 9-11 with higher molecular weights. In contrast, the addition of compound I-1b significantly reduced the signal intensity in fractions 9-11, indicating that compound I-1b did interfere C-TDP-43 oligomeric intermediates. Again, the addition of both compounds I-1b and MG132 failed to reduce the aforementioned species (FIG. 7, (B)).

Collectively, the results in the present disclosure demonstrated the dual-targeting capacity of the present compound of formula (I) or formula (II) against both protein aggregates and oligomers. Thus, the present compound of formula (I) or formula (II) could serve as a candidate compound for the development of a medicament for the treatment of neurodegenerative diseases associated with misfolded proteins.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A compound having the structure of formula (I), 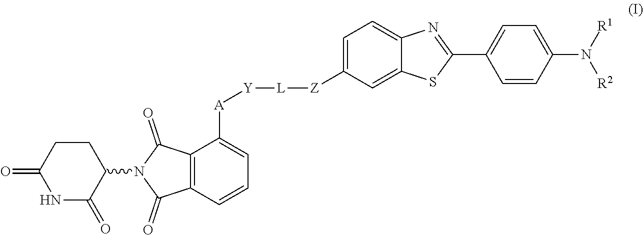
wherein,
X and Y are independently —CH₂—, —CH₂CH₂— or —C═O;
A is —CH₂— or —NH;
Z is —NH, O, or S;
L is —(CH₂CH₂)ₘ—, —(OCH₂CH₂)ₙ—, or 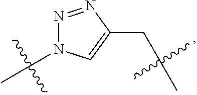
in which m and n are independently an integral between 1 to 8; and
R¹ and R² are independently H or C₁₋₆ alkyl.

2. The compound of claim 1, wherein the compound has the structure of formula (I-1) 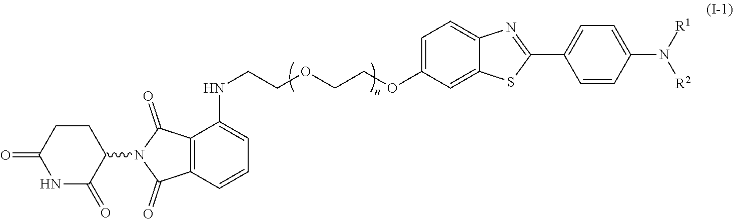
wherein,
n is an integral between 1 to 4; and
R¹ and R² are independently H or C₁₋₆ alkyl.

3. The compound of claim 2, wherein n is 2, R¹ and R² are independently —CH₃.

4. The compound of claim 1, wherein the compound has the structure of formula (I-2) 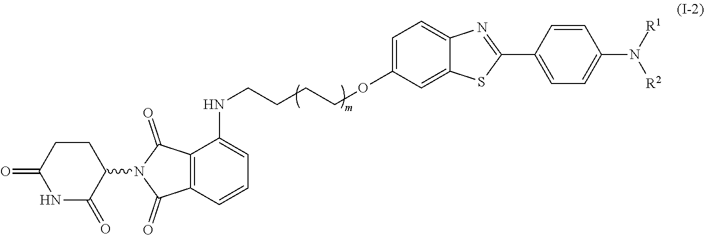
wherein,
m is an integral between 1 to 4; and
R¹ and R² are independently H or C₁₋₆ alkyl.

5. The compound of claim 1, wherein the compound has the structure of formula (I-3)

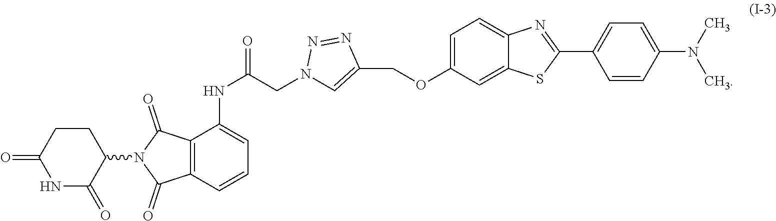 [SMILES: CN(C)c1ccc(-c2nc3ccc(OCc4cn(CC(=O)Nc5cccc6c5C(=O)N(C5CCC(=O)NC5=O)C6=O)nn4)cc3s2)cc1]

6. A compound having the structure of formula (II), 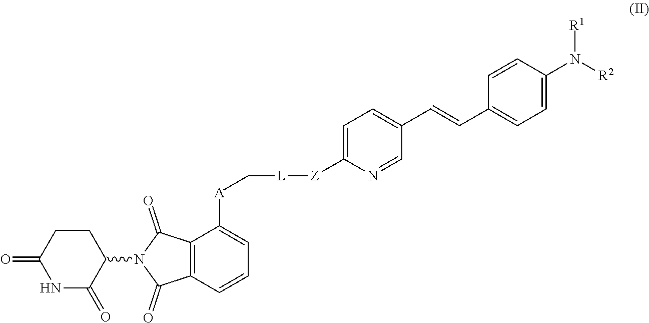
wherein,
A is —CH₂— or —NH;
Z is —NH, O, or S;
L is —(CH₂CH₂)ₘ—, or —(OCH₂CH₂)ₙ—, in which m and n are independently an integral between 1 to 8; and
R¹ and R² are independently H or C₁₋₆ alkyl.

7. The compound of claim 6, wherein the compound has the structure of formula (II-1), 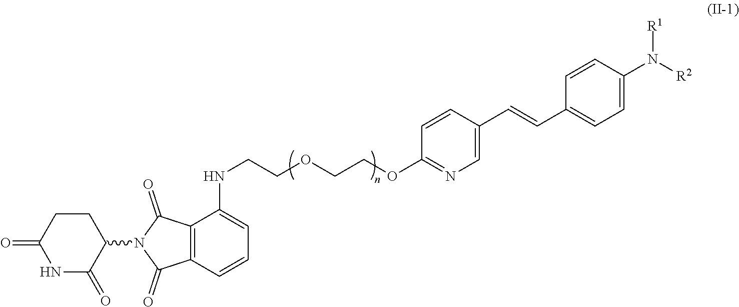
wherein,
n is an integral between 1 to 4; and
R¹ and R² are independently H or C₁₋₆ alkyl.

8. The compound of claim 7, wherein n is 4, R¹ is H and R² is —CH₃.

9. The compound of claim 6, wherein the compound has the structure of formula (II-2) 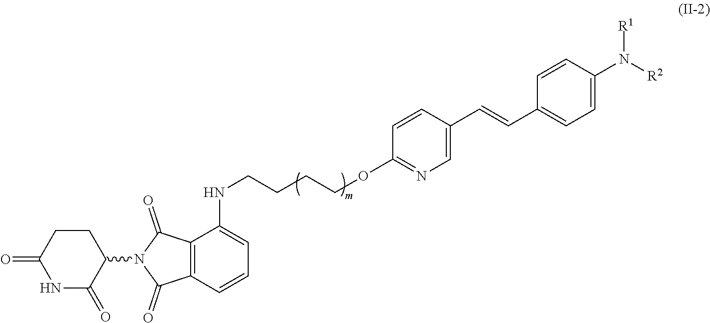
wherein,
m is an integral between 1 to 4; and
R¹ and R² are independently H or C₁₋₆ alkyl.

10. (canceled)

11. A method of treating a subject suffering from a neurodegenerative disease comprising administering to the subject an effective amount of the compound of claim 1.

12. The method of claim 11, wherein the neurodegenerative disease is selected from the group consisting of amyotrophic lateral sclerosis (ALS), frontotemporal dementia (FTD), Alzheimer's disease (AD), and Huntington's disease (HD).

13. The method of claim 11, wherein the subject is a human.
